(19) United States
(12) Patent Application Publication
(10) Pub. No.: US 2023/0284614 A1
(43) Pub. Date: Sep. 14, 2023

(54) ORGAN TRANSPORT TRACKING

(71) Applicant: Paragonix Technologies, Inc., Braintree, MA (US)

(72) Inventors: Lisa Maria Anderson, Cambridge, MA (US); Adam Lafreniere, Braintree, MA (US); Michael Tajima, Braintree, MA (US)

(21) Appl. No.: 18/119,653

(22) Filed: Mar. 9, 2023

Related U.S. Application Data
(60) Provisional application No. 63/318,970, filed on Mar. 11, 2022.

(51) Int. Cl. A01N 1/02

(52) U.S. Cl. CPC A01N 1/0252; A01N 1/0284

(57) ABSTRACT

Systems and methods for providing secure, sterile, and temperature-controlled environment for transporting biological samples and further providing active tracking allowing a medical team, or any other interested party, to know the geographic location and condition of the biological sample, as well as the state of the consumables.

### CROSS-REFERENCE TO RELATED APPLICATIONS

This application claims priority to, and the benefit of, U.S. Provisional Application No. 63/318,970, filed Mar. 11, 2022, the content of which is incorporated by reference herein in its entirety.

### FIELD OF THE INVENTION

The invention relates generally to hypothermic transport of biological samples, and, more particularly, to systems and methods for providing secure, sterile, and temperature-controlled environment for transporting biological samples and further providing active tracking allowing a medical team, or any other interested party, to know the geographic location and condition of the biological sample, as well as the state of the consumables.

### BACKGROUND

There is a critical shortage of donor organs. Hundreds of lives could be saved each day if more organs (heart, kidney, lung, etc.) were available for transplant. While the shortage is partly due to a lack of donors, there is a need for better methods of preserving and transporting donated organs. Current storage and preservation methods allow only small time windows between harvest and transplant, typically on the order of hours. These time windows dictate who is eligible to donate organs and who is eligible to receive the donated organs. These time windows also result in eligible organs going unused because they cannot be transported to a recipient in time.

The transport window is most acute for heart transplants. Current procedures dictate that hearts cannot be transplanted after four hours of ischemia (lack of blood supply). Because of this time limit, a donor heart cannot be transplanted into a recipient who is located more than 500 miles (800 km) from the harvest. In the United States, this means that a critically-ill patient in Chicago will be denied access to a matching donor heart from New York City. If the geographic range of donors could be extended, thousands of lives would be saved each year.

While several state-of-the-art preservation methods are available to keep organs viable within a hospital, transport preservation typically involves simple hypothermic (less than 10° C.) storage. Contemporary transport storage (i.e. “picnic cooler” storage) typically involves bagging the organ in cold preservation solution and placing the bagged organ in a portable cooler along with ice for the journey. There are no additional nutrients or oxygen provided to the organ. For the most part, the hope is that the preservation solution will reduce swelling and keep the tissues moist, while the cold reduces tissue damage due to hypoxia.

This method of transport has several known shortcomings, however. First, the temperature is not stabilized. Because the temperature of the organ is determined by the rate of melting and the thermal losses of the cooler, an organ will experience a wide range of temperatures during transport. For example, the temperatures can range from nearly 0° C., where the organ risks freezing damage, to 10-15° C., or greater, where the organ experiences greater tissue damage due to hypoxia.

Second, the organ does not receive sufficient oxygen and nutrients. Even though the metabolic rate is greatly slowed by the low temperatures, the tissues still require oxygen and nutrients to be able to function normally once the tissue is warmed. While some nutrients are provided by the preservation fluid surrounding the organ, the nutrients are not readily absorbed by the exterior of the organ due to the presence of a protective covering, e.g., the renal capsule.

Third, there is little protection against mechanical shock. An organ sealed in bag and then placed in a cooler with ice is subject to bruising and abrasion as the organ contacts ice chunks or the sides of the cooler. Mechanical damage can be especially problematic when the organ is airlifted and the aircraft experiences turbulence.

Fourth, there is no way to monitor the conditions during transport. Monitoring temperature and oxygen consumption, for example, would give an indication of the condition of the organ. Such information could be used by a transport team to correct conditions, e.g., add more ice, or to indicate that the organ may not be suitable for transplant. If real-time data were available, it would additionally help receiving transport teams to determine the best time to prepare the recipient for the transplant. Especially in cases of recipients with bad health, e.g., heart failure, it is paramount to minimize the amount of time that the patient is under anesthesia.

Improved transport and storage for organs would increase the pool of available organs while improving outcomes for recipients.

### SUMMARY

The disclosed system for hypothermic transport overcomes the shortcomings of the prior art by providing a sterile, temperature-stabilized environment for the samples while providing the ability to monitor the location and conditions of the tissue during transport. User applications can be accessed on any computing device (e.g., phones, tablets, or desktops) and provide real-time, centralized, secure coordination for transplant teams including pairing with organ transport systems to share organ status with the entire team. In various embodiments, a web-based portal can provide digital participation in live sessions or review of historical case data. Real-time temperature data can be provided to connect teams across any distance.

Computer-based programs described herein can provide real-time information such as organ status, location, and case status as well as allow for secure communication among the transplant team. In various embodiments, Bluetooth pairing with an organ preservation system as described herein (or commercially available, for example, from Paragonix Technologies, Inc., Cambridge, Mass.) to allow all registered or connected team members to track the organ conditions such as organ temperature and ambient temperature. Global Positions System (GPS) tracking of the procurement team en route to and from the donor center can also be provided from GPS sensors in or on the organ preservation system or by utilizing the GPS sensors located in a selected mobile device among the transport team. HIPAA compliant messaging and communications can be provided in the application to keep the procurement team, OPO, donor hospital, and recipient team informed. The dedicated organ transport application can provide a centralized hub to allow members from different hospital systems and organizations to communicate in a secure manner as opposed to relying on incompatible internal communications systems among, for example, a donor hospital, a recipient hospital, and a transport team. At-a-glance graphic status trackers can be used to provide a snapshot summary of timing of key events in the transplant.

In various embodiments, systems and methods of the invention may include creating a session for a specific organ transport case. A user can initiate a session and invite other users/team members to join. A session key may be provided and required to ensure secure access to the application. In creating a session, a user may select a type of device such as those available from Paragonix Technologies, Inc. and may be provided with visual ques such as a picture of the various devices in order to simplify correct identification and selection of transport device. The user can also enter a type of donation (e.g., heart or lung) and a session type (e.g., a clinical case or training session).

Systems and methods of the invention can accordingly increase efficiencies by reducing time-sensitive phone calls to multiple parties and providing a single secure platform to allow users to reach everyone on the case simultaneously. Systems and methods can also provide increased team connectivity as, regardless of location, a team can track the organ's status and location and the clinical team can reduce down-time by being alerted to when their participation is required. Furthermore, systems and methods of the invention can allow users to meet quality goals by creating a permanent record of clinical and logistical milestones and providing organ status data downloads for quality, research and patient record purposes.

Additionally, because the samples are suspended in an oxygenated preservation fluid, the delivered samples avoid mechanical damage, remain oxygenated, and are delivered healthier than samples that have been merely sealed in a plastic bag.

In some cases in which the sample is a tissue, the preservation solution is circulated through the tissue using the tissue's cardiovascular system. In this case, a pulsed flow is used to imitate the natural environment of the tissue. Such conditions improve absorption of nutrients and oxygen as compared to static storage. Additionally, because compressed oxygen is used to propel the pulsed circulation, the preservation fluid is reoxygenated during transport, replacing the oxygen that has been consumed by the tissue and displacing waste gases (i.e., CO₂). In some instances, a suite of sensors measures temperature, oxygen content, and pressure of the circulating fluids to assure that the tissue experiences a favorable environment during the entire transport.

In one version of the invention, the system includes a first transport container configured to suspend a biological sample (e.g., tissue or an organ) in a preservation fluid. The first transport container includes a temperature sensor, thereby allowing a user to continually monitor the temperature of the tissue. The system also includes a second transport container having an insulated cavity for receiving the first transport container, and having recesses for receiving cooling media. The second transport container may additionally have a display for displaying the temperature. In an embodiment, the second transport container included a positioning receiver and a positioning transporter, thereby allowing real-time tracking of the position of the container. This information can be accessed by a transport team via a website, mobile device, tablet, or pager.

In another version of the invention, the system includes a first transport container that has a pumping chamber to circulate a fluid inside the first transport container. The first transport container includes a temperature sensor and a temperature display, thereby allowing a user to continually monitor the temperature of the tissue. The system also includes a second transport container having an insulated cavity for receiving the first transport container and having recesses for receiving cooling media. The second transport container may additionally have a display for displaying the temperature. In an embodiment, the second transport container included a positioning receiver and a positioning transporter, thereby allowing real-time tracking of the position of the container. This information can be accessed by a transport team via a website, mobile device, tablet, or pager.

Typically, the cooling media will be one or more eutectic cooling blocks. The cooling blocks provide regulated cooling in the range of 4-8° C. for twelve or more hours. The system may additionally include an oxygen source, for example a compressed gas cylinder, to provide oxygen to the biological sample. In some versions, the system will have sensors and displays to monitor conditions in addition to temperature, for example oxygen flow, oxygen consumption, or pressure. In some versions, the sensors that monitor, for example, the temperature of the sample, will be coupled to a wireless transmitter that communicates with a second display located on the exterior of the second transport container. Accordingly, a user can monitor the temperature of the biological sample within the first transport container while the first transport container is securely stored within the second transport container. The pressure, temperature, and flow data may also be transmitted from a wireless transmitter incorporated into the second transport container. In other embodiments, the oxygen source may include a sensor for monitoring the pressure in the oxygen source, e.g., an oxygen cylinder. The pressure of the oxygen source may additionally be transmitted from the transmitter incorporated into the second transport container.

### BRIEF DESCRIPTION OF THE DRAWINGS

Features and advantages of the claimed subject matter will be apparent from the following detailed description of embodiments consistent therewith, which description should be considered with reference to the accompanying drawings.

FIG. 1 shows an embodiment of a first transport container suitable for use as part of a hypothermic transport system of the invention.

FIG. 2 is a perspective view of a first transport container suitable for use with a hypothermic transport system of the invention.

FIG. 3 is a cross-sectional view of a first transport container suitable for use with a hypothermic transport system of the invention. The lid of the container comprises a pumping chamber for circulating or perfusing a preservation solution.

FIG. 4 is a schematic representation of a donor heart suspended in a first transport container and being perfused with oxygenated preservation solution.

FIG. 5 shows an embodiment of a hypothermic transport system of the invention, including a first transport container, a second transport container, and cooling media for maintaining the temperature of the tissue being transported. The first transport container comprises a temperature sensor and a display, and the temperature can be wirelessly communicated to a second display on the exterior of the second transport container.

FIG. 6 shows an embodiment of a hypothermic transport system of the invention, including a first transport container, a second transport container, and recesses for holding cooling media for maintaining the temperature of the tissue being transported. The second transport container is also configured to transport a source of oxygen.

FIG. 7 shows a cut-away view of a hypothermic transport system of the invention, with detail of the interior structures that provide additional mechanical protection to the first transport container and its contents.

FIG. 8 shows an embodiment of a hypothermic transport system of the invention, including a first transport container, a second transport container, a source of oxygen, sensors for sensing the pressure within the source of oxygen, a parameter display, and a position receiver/transmitter.

FIG. 9A shows an embodiment of a hypothermic transport system of the invention, including a first transport container, a second transport container, a source of oxygen, sensors for sensing the pressure within the source of oxygen, a parameter display, and a position receiver/transmitter.

FIG. 9B shows a cut away of the pieces of FIG. 9A assembled for transport.

FIG. 10 is a flow chart detailing a how organ and transport information can be used to determine whether a transport procedure should proceed.

FIG. 11 illustrates that the system is configured to provide position and/or condition parameters to a distributed network during various phases of tissue transport, including air, ground, and local transport.

FIG. 12 is a flow chart showing an embodiment of a transport system that is configured to switch to flight track mode when the transport apparatus enters the signal field of an airport.

FIG. 13 illustrates a computer terminal or web page where information about the position and condition of the tissue can be accessed during transport.

FIG. 14 illustrates a mobile phone or tablet where information about the position and condition of the tissue can be accessed during transport.

FIG. 15 illustrates a pager message that may be generated by the system based upon the position and condition of the tissue.

FIG. 16 shows an exemplary user interface displaying organ status information.

FIG. 17 shows an exemplary user interface displaying organ location.

FIG. 18 shows an exemplary user interface displaying case status.

FIG. 19 shows an exemplary user interface displaying case team communications.

FIG. 20 shows exemplary user interfaces for session creation.

FIG. 21 shows an exemplary home screen for a user interface.

FIG. 22 shows an exemplary user interface for device selection in creating a session.

FIG. 23 shows an exemplary user interface for donation selection in creating a session.

FIG. 24 shows an exemplary user interface for session type selection.

FIG. 25 shows an exemplary user interface requesting notification permissions.

FIG. 26 shows an exemplary user interface for joining an existing session.

FIG. 27 shows an exemplary user interface displaying additional organ status information.

FIG. 28 shows an exemplary user interface displaying member information.

FIG. 29 shows an exemplary user interface displaying a data logger start screen.

### DETAILED DESCRIPTION

The disclosed systems for hypothermic transport of samples provides a sterile, temperature-stabilized environment for transporting samples while providing an ability to monitor the temperature of the samples during transport. Because of these improvements, users of the invention can reliably transport samples over much greater distances, thereby substantially increasing the pool of available tissue donations. Additionally, because the tissues are in better condition upon delivery, the long-term prognosis for the recipient is improved. The system provides real-time data to assist receiving transport teams in determining the best time to prepare a recipient for a transplant. In the event that the organ expires, the transplant team will know not to being preparing the recipient.

Hypothermic transport systems of the invention comprise a first transport container and a second transport container. The first transport container will receive the tissue for transport, and keep it suspended or otherwise supported in a surrounding pool of preservation solution. The first transport container may comprise a number of configurations suitable to transport tissues hypothermicly, provided that the first transport container includes a temperature sensor and a display. For example, the first transport container could be of a type disclosed in U.S. Pat. Nos. 8,785,116, and 8,828,710, and 8,835,158, all of which are incorporated by reference herein in their entireties.

In some embodiments, the first transport container will include a pumping mechanism to circulate the preservation solution or perfuse an organ with the preservation solution. A first transport container comprising a pumping chamber will be referred to as “pulsatile.” While the pumping is pulsating in preferred embodiments, the pumping is not intended to be limited to pulsating pumping, that is, the pumping may be continuous. In other embodiments, the first transport container will not circulate or perfuse the preservation solution. A non-pumping first transport container will be referred to as “static.”

In some instances the first transport container will be a static transport container. The static first transport container includes a storage vessel and a lid without a pumping chamber. The lid without a pumping chamber is coupled to an adapter which can be used to suspend a tissue to be transported. The adapter can be coupled to the tissue T in any suitable manner. It should be noted that the tissue T shown in the figures is for illustrative purposes only. That is, the invention is intended for the transport of biological samples, generally, which may include tissues, organs, body fluids, and combinations thereof.

The static first transport container also includes a temperature sensor which is coupled to a temperature display disposed on the exterior of the static first transport container. While the temperature display is shown disposed on the exterior of the lid, it could also be disposed on the exterior of the storage vessel. Typically, the tissue will be affixed to the adapter, coupled to the lid, and then the lid and the tissue T will be immersed into preservation solution held by storage vessel. The lid will then be sealed to the storage vessel using a coupling. In some embodiments, the lid or the storage vessel will have entrance and exit ports to allow a user to purge the sealed static first transport container by forcing additional preservation fluid into the sealed container.

The storage vessel, lid without a pumping chamber, and adapter are constructed of durable materials that are suitable for use with a medical device. Additionally, the transport container should be constructed of materials that conduct heat so that the sample within the container is adequately cooled by the cooling media (see discussion below). For example, the lid and storage vessel may be constructed of stainless steel. In other embodiments, because it is beneficial to be able to view the contents directly, the lid and storage vessel may be constructed of medical acrylic (e.g., PMMA) or another clear medical polymer.

It is additionally beneficial for the storage vessel, lid without a pumping chamber, and adapter to be sterilizable, i.e., made of a material that can be sterilized by steam (autoclave) or with UV irradiation, or another form of sterilization. Sterilization will prevent tissues from becoming infected with viruses, bacteria, etc., during transport. In a typical embodiment the first transport container will be delivered in a sterile condition and sealed in sterile packaging. In some embodiments, the first transport container will be sterilized after use prior to reuse, for example a hospital. In other embodiments, the first transport container will be disposable.

The temperature sensor may be any temperature reading device that can be sterilized and maintained in cold fluidic environment, i.e., the environment within the static first transport container 1 during transport of tissue. The temperature sensor may be a thermocouple, thermistor, infrared thermometer, or liquid crystal thermometer. When the static first transport container is sealed, temperature sensor is typically disposed in contact with the cold preservation solution and in proximity to the tissue such that a temperature of the tissue can be ascertained during transport. Temperature display may be coupled to the temperature sensor using any suitable method, for example a wire, cable, connector, or wirelessly using available wireless protocols. In some embodiments, the temperature sensor may be attached to the adapter. In some embodiment, the temperature sensor is incorporated into the adapter to improve the mechanical stability of the temperature sensor.

The temperature display can be any display suitable for displaying a temperature measured by the temperature sensor or otherwise providing information about the temperature within the static first transport container 1. For example, the temperature display can be a light emitting diode (LED) display or liquid crystal display (LCD) showing digits corresponding to a measured temperature. The display may alternatively comprise one or more indicator lights, for example an LED which turns on or off or flashes to indicated whether the temperature measured by the temperature sensor is within an acceptable range, e.g., 2-8° C., e.g., 4-6° C., e.g., about 4° C. The temperature sensor may also be connected to a processor (not shown) which will compare the measured temperature to a threshold or range and create an alert signal when the temperature exceeds the threshold or range. The alert may comprise an audible tone, or may signal to a networked device, e.g., a computer, cell phone, or pager that the temperature within the container exceeds the desired threshold or range.

The adapter may be of a variety of structures suitable to suspend the tissue in the preservation solution while minimizing the potential for mechanical damage, e.g., bruising or abrasion. In some embodiments, the adapter is configured to be sutured to the tissue. In another example, the adapter is coupleable to the tissue via an intervening structure, such as silastic or other tubing. In some embodiments, at least a portion of the adapter, or the intervening structure, is configured to be inserted into the tissue. In some embodiments, the adapter is configured to support the tissue when the tissue is coupled to the adapter. For example, in some embodiments, the adapter includes a retention mechanism configured to be disposed about at least a portion of the tissue and to help retain the tissue with respect to the adapter. The retention mechanism can be, for example, a net, a cage, a sling, or the like.

In some embodiments, a first transport container may additionally include a basket or other support mechanism configured to support the tissue when the tissue is coupled to the adapter or otherwise suspended in the first transport container. The support mechanism may be part of an insert which fits within the first transport container. The basket may include connectors which may be flexible or hinged to allow the basket to move in response to mechanical shock, thereby reducing the possibility of damage to tissue. In other embodiments, the basket may be coupled to the lid so that it is easily immersed in and retracted from the preservation fluid held in the storage vessel.

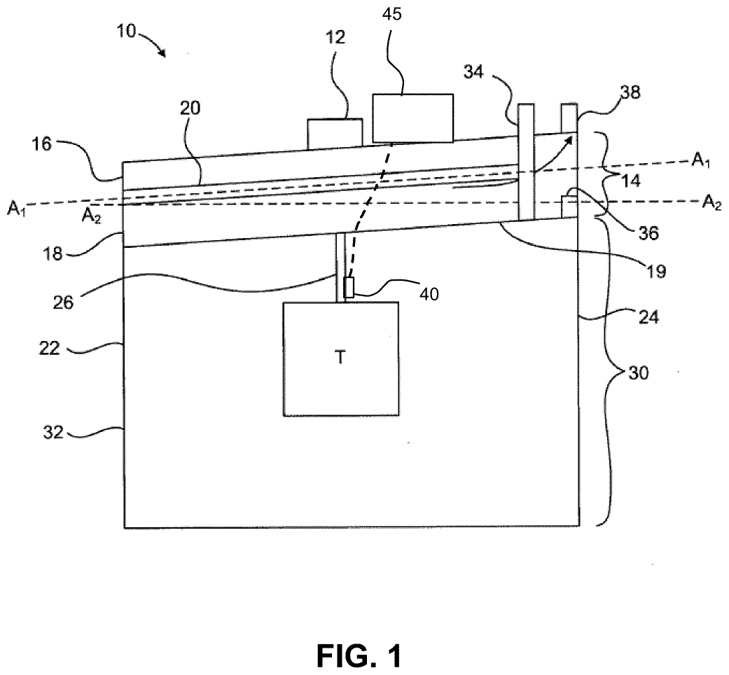

In some instances, the first transport container will be equipped to pump or circulate the preservation fluid. A pulsatile first transport container 10 is shown in FIG. 1. The pulsatile first transport container 10 is configured to oxygenate a preservation fluid received in a pumping chamber 14 of the apparatus. The pulsatile first transport container 10 includes a valve 12 configured to permit a fluid (e.g., oxygen) to be introduced into a first portion 16 of the pumping chamber 14. A membrane 20 is disposed between the first portion 16 of the pumping chamber 14 and a second portion 18 of the pumping chamber. The membrane 20 is configured to permit the flow of a gas between the first portion 16 of the pumping chamber 14 and the second portion 18 of the pumping chamber through the membrane. The membrane 20 is configured to substantially prevent the flow of a liquid between the second portion 18 of the pumping chamber 14 and the first portion 16 of the pumping chamber through the membrane. In this manner, the membrane can be characterized as being semi-permeable.

The membrane 20 is disposed within the pumping chamber 14 along an axis A1 that is transverse to a horizontal axis A2. Said another way, the membrane 20 is inclined, for example, from a first side 22 to a second side 24 of the apparatus 10. As such, a rising fluid in the second portion 18 of the pumping chamber 14 will be directed by the inclined membrane 20 toward a port 38 disposed at the highest portion of the pumping chamber 14. The port 38 is configured to permit the fluid to flow from the pumping chamber 14 into the atmosphere external to the apparatus 10. In some embodiments, the port 38 is configured for unidirectional flow, and thus is configured to prevent a fluid from being introduced into the pumping chamber 14 via the port (e.g., from a source external to the pulsatile first transport container 10). In some embodiments, the port 38 includes a luer lock.

The second portion 18 of the pumping chamber 14 is configured to receive a fluid. In some embodiments, for example, the second portion 18 of the pumping chamber 14 is configured to receive a preservation fluid. The second portion 18 of the pumping chamber 14 is in fluid communication with the adapter 26. In pulsatile first transport container 10, the adapter 26 is configured to permit movement of the fluid from the pumping chamber 14 to a tissue T. In some embodiments, the pumping chamber 14 defines an aperture configured to be in fluidic communication with a lumen (not shown) of the adapter 26. The adapter 26 is configured to be coupled to the tissue T. The adapter 26 can be coupled to the tissue T in any suitable manner. For example, in some embodiments, the adapter 26 is configured to be sutured to the tissue T. In another example, the adapter 26 is coupleable to the tissue T via an intervening structure, such as silastic or other tubing. In some embodiments, at least a portion of the adapter 26, or the intervening structure, is configured to be inserted into the tissue T. For example, in some embodiments, the lumen of the adapter 26 (or a lumen of the intervening structure) is configured to be fluidically coupled to a vessel of the tissue T. In other embodiments, the tissue T may be suspended in a basket 8 and not connected to the adapter 26. In these embodiments, the pumping chamber serves to circulate the preservation fluid, however the tissue T is not perfused. In some embodiments, the adapter 26 is configured to support the tissue T when the tissue T is coupled to the adapter. For example, in some embodiments, the adapter 26 includes a retention mechanism (not shown) configured to be disposed about at least a portion of the tissue T and to help retain the tissue T with respect to the adapter. The retention mechanism can be, for example, a net, a cage, a sling, or the like.

An organ chamber 30 is configured to receive the tissue T and a fluid. In some embodiments, the pulsatile first transport container 10 includes a port 34 that is extended through the pulsatile first transport container 10 (e.g., through the pumping chamber 14) to the organ chamber 30. The port 34 is configured to permit fluid (e.g., preservation fluid) to be introduced to the organ chamber 30. In this manner, fluid can be introduced into the organ chamber 30 as desired by an operator of the apparatus. For example, in some embodiments, a desired amount of preservation fluid is introduced into the organ chamber 30 via the port 34, such as before disposing the tissue T in the organ chamber 30 and/or while the tissue T is received in the organ chamber. In some embodiments, the port 34 is a unidirectional port, and thus is configured to prevent the flow of fluid from the organ chamber 30 to an area external to the organ chamber through the port. In some embodiments, the port 34 includes a luer lock. The organ chamber 30 may be of any suitable volume necessary for receiving the tissue T and a requisite amount of fluid for maintaining viability of the tissue T. In one embodiment, for example, the volume of the organ chamber 30 is approximately 2 liters.

The organ chamber 30 is formed by a canister 32 and a bottom portion 19 of the pumping chamber 14. In a similar manner as described above with respect to the membrane 20, an upper portion of the organ chamber (defined by the bottom portion 19 of the pumping chamber 14) can be inclined from the first side 22 towards the second side 24 of the apparatus. In this manner, a rising fluid in the organ chamber 30 will be directed by the inclined upper portion of the organ chamber towards a valve 36 disposed at a highest portion of the organ chamber. The valve 36 is configured to permit a fluid to flow from the organ chamber 30 to the pumping chamber 14. The valve 36 is configured to prevent flow of a fluid from the pumping chamber 14 to the organ chamber. The valve 36 can be any suitable valve for permitting unidirectional flow of the fluid, including, for example, a ball check valve.

The canister 32 can be constructed of any suitable material. In some embodiments, the canister 32 is constructed of a material that permits an operator of the pulsatile first transport container 10 to view at least one of the tissue T or the preservation fluid received in the organ chamber 30. For example, in some embodiments, the canister 32 is substantially transparent. In another example, in some embodiments, the canister 32 is substantially translucent. The organ chamber 30 can be of any suitable shape and/or size. For example, in some embodiments, the organ chamber 30 can have a perimeter that is substantially oblong, oval, round, square, rectangular, cylindrical, or another suitable shape.

Like the static first transport container 1, a pulsatile first transport container 10 also includes a temperature sensor 40 which is coupled to a temperature display 45 disposed on the exterior of the pulsatile first transport container 10. While the temperature display 45 is shown disposed on the pumping chamber 14, it could also be disposed on the canister 32. Typically, the tissue T will be affixed to the adapter 26, coupled to the pumping chamber 14, and then the pumping chamber 14 and the tissue T will be immersed into preservation solution held by organ chamber 30.

The temperature sensor 40 may be any temperature reading device that can be sterilized and maintained in cold fluidic environment, i.e., the environment within the static first transport container 1 during transport of tissue T. The temperature sensor 40 may be a thermocouple, thermistor, infrared thermometer, or liquid crystal thermometer. When the static first transport container 1 is sealed, temperature sensor 40 is typically disposed in contact with the cold preservation solution and in proximity to the tissue T such that a temperature of the tissue T can be ascertained during transport. Temperature display 45 may be coupled to the temperature sensor 40 using any suitable method, for example a wire, cable, connector, or wirelessly using available wireless protocols. In some embodiments, the temperature sensor 40 may be attached to the adapter 26. In some embodiment, the temperature sensor 40 is incorporated into the adapter 26 to improve the mechanical stability of the temperature sensor 40.

The temperature display 45 can be any display suitable for displaying a temperature measured by the temperature sensor 40, or otherwise providing information about the temperature within the pulsatile first transport container 10. For example, the temperature display can be a light emitting diode (LED) display or liquid crystal display (LCD) showing digits corresponding to a measured temperature. The display may alternatively comprise one or more indicator lights, for example an LED which turns on or off or flashes to indicate whether the temperature of measured by the temperature sensor 40 is within an acceptable range, e.g., 2-8° C., e.g., 4-6° C., e.g., about 4° C. The temperature sensor 40 may also be connected to a processor (not shown) which will compare the measured temperature to a threshold or range and create an alert signal when the temperature exceeds the threshold or range. The alert may comprise an audible tone, or may signal to a networked device, e.g., a computer, cell phone, or pager that the temperature within the container exceeds the desired threshold or range.

In use, the tissue T is coupled to the adapter 26. The pumping chamber 14 is coupled to the canister 32 such that the tissue T is received in the organ chamber 30. In some embodiments, the pumping chamber 14 and the canister 32 are coupled such that the organ chamber 30 is hermetically sealed. A desired amount of preservation fluid is introduced into the organ chamber 30 via the port 34. The organ chamber 30 can be filled with the preservation fluid such that the preservation fluid volume rises to the highest portion of the organ chamber. The organ chamber 30 can be filled with an additional amount of preservation fluid such that the preservation fluid flows from the organ chamber 30 through the valve 36 into the second portion 18 of the pumping chamber 14. The organ chamber 30 can continue to be filled with additional preservation fluid until all atmospheric gas that initially filled the second portion 18 of the pumping chamber 14 rises along the inclined membrane 20 and escapes through the port 38. Because the gas will be expelled from the pumping chamber 14 via the port 38 before any excess preservation fluid is expelled (due to gas being lighter, and thus more easily expelled, than liquid), an operator of the pulsatile first transport container 10 can determine that substantially all excess gas has been expelled from the pumping chamber when excess preservation fluid is released via the port. As such, the pulsatile first transport container 10 can be characterized as self-purging.

Oxygen (or another suitable fluid, e.g., dry air) is introduced into the first portion 16 of the pumping chamber 14 via the valve 12. A positive pressure generated by the introduction of oxygen into the pumping chamber 14 causes the oxygen to be diffused through the semi-permeable membrane 20 into the second portion 18 of the pumping chamber. Because oxygen is a gas, the oxygen expands to substantially fill the first portion 16 of the pumping chamber 14. As such, substantially the entire surface area of the membrane 20 between the first portion 16 and the second portion 18 of the pumping chamber 14 is used to diffuse the oxygen. The oxygen is diffused through the membrane 20 into the preservation fluid received in the second portion 18 of the pumping chamber 14, thereby oxygenating the preservation fluid.

In the presence of the positive pressure, the oxygenated preservation fluid is moved from the second portion 18 of the pumping chamber 14 into the tissue T via the adapter 26. For example, the positive pressure can cause the preservation fluid to move from the pumping chamber 14 through the lumen of the adapter 26 into the vessel of the tissue T. The positive pressure is also configured to help move the preservation fluid through the tissue T such that the tissue T is perfused with oxygenated preservation fluid.

After the preservation fluid is perfused through the tissue T, the preservation fluid is received in the organ chamber 30. In this manner, the preservation fluid that has been perfused through the tissue T is combined with preservation fluid previously disposed in the organ chamber 30. In some embodiments, the volume of preservation fluid received from the tissue T following perfusion combined with the volume of preservation fluid previously disposed in the organ chamber 30 exceeds a volume (e.g., a maximum fluid capacity) of the organ chamber 30. A portion of the organ chamber 30 is flexible and expands to accept this excess volume. The valve 12 can then allow oxygen to vent from the first portion 16 of the pumping chamber 14, thus, reducing the pressure in the pumping chamber 14. As the pressure in the pumping chamber 14 drops, the flexible portion of the organ chamber 30 relaxes, and the excess preservation fluid is moved through the valve 36 into the pumping chamber 14. The cycle of oxygenating preservation fluid and perfusing the tissue T with the oxygenated reservation fluid can be repeated as desired.

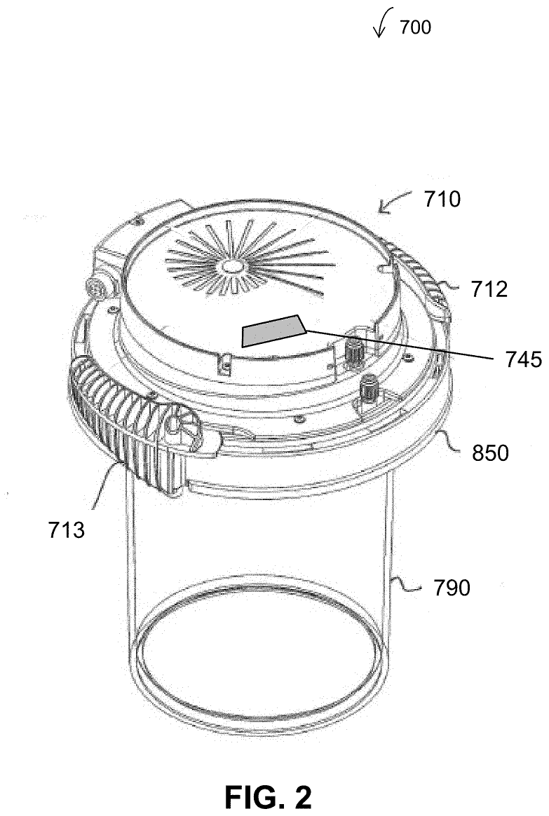

A perspective view of a first transport container suitable for use as a portion of a system of the invention is shown in FIG. 2. First transport container 700 comprises a lid assembly 710 having a temperature display 745, a canister 790, and a coupling mechanism 850 between the lid 710 and the canister 790. The first transport container 700 may be hermetically sealed by actuating clamps 712 and 713, sealing the coupling mechanism 850, once the tissue and preservation fluid has been placed within. As shown in FIG. 2, the canister may be substantially transparent, allowing a user to view the condition of the tissue during transport.

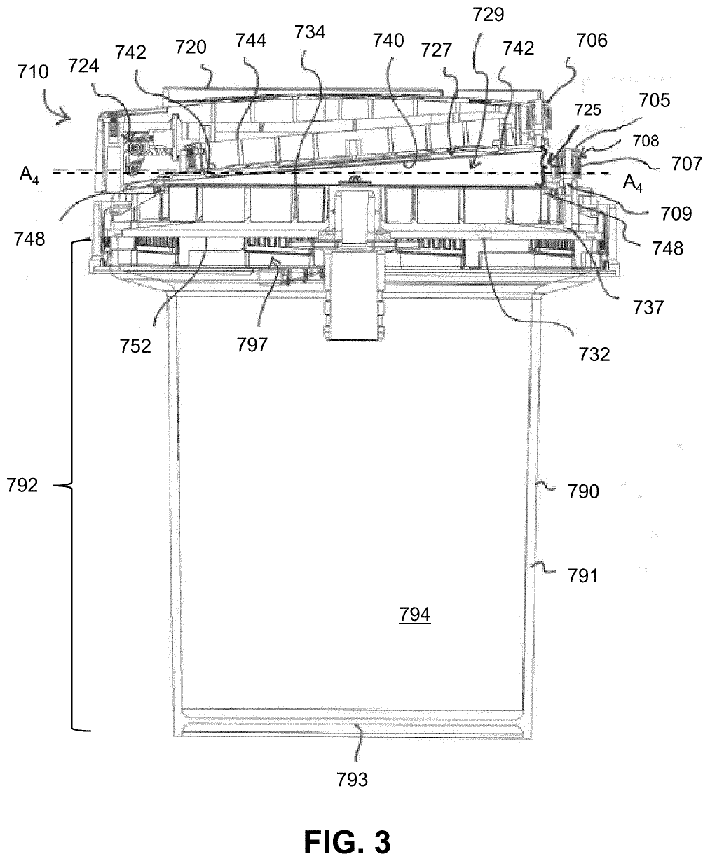

A cut-away view of first transport container capable of perfusing an organ with preservation fluid is shown in FIG. 3. It includes a lid assembly 710, a canister 790, and a coupling mechanism 850. While it is not shown in this view, the first transport container additionally comprises a temperature sensor and a display. The lid assembly 710 includes a fill port 708 configured to permit introduction of a fluid (e.g., the perfusate) into an organ/storage chamber 792 (e.g., when the lid assembly 710 is coupled to the canister 790). The fill port 708 can be similar in many respects to a port described herein (e.g., port 74, fill port 108). In the embodiment illustrated in FIG. 3, the fill port 708 includes a fitting 707 coupled to the lid 720 and defines a lumen 709 in fluidic communication with a lumen 737 defined by the base 732, which lumen 737 is in fluidic communication with the organ chamber 792. The fitting 707 can be any suitable fitting, including, but not limited to, a luer lock fitting. The fill port 708 can include a cap 705 removably coupled to the port. The cap 705 can help prevent inadvertent movement of fluid, contaminants, or the like through the fill port 708.

The lid assembly 710 defines a chamber 724 configured to receive components of a pneumatic system (not shown) and necessary control electronics. In some embodiments, the chamber 724 is formed by a lid 720 of the lid assembly 710. In some embodiments, the chamber 724 can be formed between a lower portion 723 of the lid 720 and an upper portion 722 of the lid. In some embodiments the canister 790 is configured to receive a basket 8, such as shown in FIG. 2.

The lid assembly 710 defines a pumping chamber 725 configured to receive oxygen to facilitate diffusion of the oxygen into a preservation fluid (not shown) and to facilitate movement of the oxygenated preservation fluid throughout the storage container. A top of the pumping chamber 725 is formed by a lower portion 728 of a membrane frame 744 of the lid assembly 710. A bottom of the pumping chamber 725 is formed by an upper surface 734 of a base 732 of the lid assembly 710.

The lid assembly 710 may include a first gasket 742, a membrane 740, and the membrane frame 744. The membrane 740 is disposed within the pumping chamber 725 and divides the pumping chamber 725 into a first portion 727 and a second portion 729 different than the first portion. The first gasket 742 is disposed between the membrane 740 and the membrane frame 744 such that the first gasket is engaged with an upper surface 741 of the membrane 740 and a lower, perimeter portion of the membrane frame 744. The first gasket 742 is configured to seal a perimeter of the first portion 727 of the pumping chamber 725 twined between the lower portion 728 of the membrane frame 744 and the upper surface 741 of the membrane 740. In other words, the first gasket 742 is configured to substantially prevent lateral escape of oxygen from the first portion 727 of the pumping chamber 725 to a different portion of the pumping chamber. In the embodiment illustrated in FIG. 3, the first gasket 742 has a perimeter substantially similar in shape to a perimeter defined by the membrane 740 (e.g., when the membrane is disposed on the membrane frame 744). In other embodiments, however, a first gasket can have another suitable shape for sealing a first portion of a pumping chamber configured to receive oxygen from a pneumatic system.

The first gasket 742 can be constructed of any suitable material. In some embodiments, for example, the first gasket 742 is constructed of silicone, an elastomer, or the like. The first gasket 742 can have any suitable thickness. For example, in some embodiments, the first gasket 742 has a thickness within a range of about 0.1 inches to about 0.15 inches. More specifically, in some embodiments, the first gasket 742 has a thickness of about 0.139 inches. The first gasket 742 can have any suitable level of compression configured to maintain the seal about the first portion 727 of the pumping chamber 725 when the components of the lid assembly 710 are assembled. For example, in some embodiments, the first gasket 742 is configured to be compressed by about 20 percent.

The membrane 740 is configured to permit diffusion of gas (e.g., oxygen) from the first portion 727 of the pumping chamber 725 through the membrane to the second portion 729 of the pumping chamber, and vice versa. The membrane 740 is configured to substantially prevent a liquid (e.g., the preservation fluid) from passing through the membrane. In this manner, the membrane 740 can be characterized as being semi-permeable. The membrane frame 744 is configured to support the membrane 740 (e.g., during the oxygenation of the preservation fluid and perfusion of the tissue). The membrane frame 744 can have a substantially round or circular shaped perimeter. The membrane frame 744 includes a first port 749A and a second port 749B. The first port 749A is configured to convey fluid between the first portion 727 of the pumping chamber and the pneumatic system (not shown). For example, the first port 749A can be configured to convey oxygen from the pneumatic system to the first portion 727 of the pumping chamber 725. The second port 749B is configured to permit a pressure sensor line (not shown) to be disposed therethrough. The pressure sensor line can be, for example, polyurethane tubing. The ports 749A, 749B can be disposed at any suitable location on the membrane frame 744, including, for example, towards a center of the membrane frame 744. Although the ports 749A, 749B are shown in close proximity, in other embodiments, the ports 749A, 749B can be differently spaced (e.g., closer together or further apart).

At least a portion of the membrane 740 is disposed (e.g., wrapped) about at least a portion of the membrane frame 744. In some embodiments, the membrane 740 is stretched when it is disposed on the membrane frame 744. The membrane 740 is disposed about a lower edge or rim of the membrane frame 744 and over at least a portion of an outer perimeter of the membrane frame 744 such that the membrane 740 is engaged with a series of protrusions (e.g., protrusion 745) configured to help retain the membrane with respect to the membrane frame. The membrane frame 744 is configured to be received in a recess 747 defined by the lid 720. As such, the membrane 740 is engaged between the membrane frame 744 and the lid 720, which facilitates retention of the membrane with respect to the membrane frame. In some embodiments, the first gasket 742 also helps to maintain the membrane 740 with respect to the membrane frame 744 because the first gasket is compressed against the membrane between the membrane frame 744 and the lid 720.

As illustrated in FIG. 3, the membrane 740 is disposed within the pumping chamber 725 at an angle with respect to a horizontal axis A4. In this manner, the membrane 740 is configured to facilitate movement of fluid towards a purge port 706 in fluid communication with the pumping chamber 725, as described in more detail herein. The angle of incline of the membrane 740 can be of any suitable value to allow fluid (e.g., gas bubbles, excess liquid) to flow towards the purge port 706 and exit the pumping chamber 725. In some embodiments, the angle of incline is approximately in the range of 1°-10°, in the range of 2°-6°, in the range of 2.5°-5°, in the range of 4°-5° or any angle of incline in the range of 1°-10° (e.g., approximately 1°, 2°, 3°, 4°, 5°, 6°, 7°, 8°, 9°, 10°). More specifically, in some embodiments, the angle of incline is approximately 5°.

The membrane 740 can be of any suitable size and/or thickness, including, for example, a size and/or thickness described with respect to another membrane herein (e.g., membrane 140). The membrane 740 can be constructed of any suitable material. For example, in some embodiments, the membrane is constructed of silicone, plastic, or another suitable material. In some embodiments, the membrane is flexible. The membrane 740 can be substantially seamless. In this manner, the membrane 740 is configured to be more resistant to being torn or otherwise damaged in the presence of a flexural stress caused by a change in pressure in the pumping chamber due to the inflow and/or release of oxygen or another gas.

The lid 720 includes the purge port 706 disposed at the highest portion of the pumping chamber 725 (e.g., at the highest portion or point of the second portion 729 of the pumping chamber 725). The purge port 706 is configured to permit movement of fluid from the pumping chamber 725 to an area external to the first transport container 700. The purge port 706 can be similar in many respects to a purge port described herein (e.g., port 78, purge ports 106, 306).

Optionally, a desired amount of preservation fluid can be disposed within the compartment 794 of the canister 790 prior to disposing the lid assembly 710 on the canister. For example, in some embodiments, a preservation fluid line (not shown) is connected to the storage chamber 792 and the device is flushed with preservation fluid, thereby checking for leaks and partially filling the canister 790 with preservation fluid. Optionally, when the canister 790 is substantially filled, the preservation fluid line can be disconnected. The lid assembly 710 is disposed on the canister 790 such that the body fluids, held by holder 726, are immersed in the storage chamber 792. The lid assembly 710 is coupled to the canister 790. Optionally, the lid assembly 710 and the canister 790 are coupled via the retainer ring 850. Optionally, a desired amount of preservation fluid is delivered to the storage chamber 792 via the fill port 708. In some embodiments, a volume of preservation fluid greater than a volume of the storage chamber 792 is delivered to the storage chamber such that the preservation fluid will move through the valves 738A, 738B into the second portion 729 of the pumping chamber 725.

In the embodiment shown in FIG. 3, oxygen may be introduced into the first portion 727 of the pumping chamber 725 via a pneumatic system. The pneumatic system is configured to generate a positive pressure by the introduction of oxygen into the first portion 727 of the pumping chamber 725. The positive pressure helps to facilitate diffusion of the oxygen through the membrane 740. The oxygen is diffused through the membrane 740 into the preservation solution disposed in the second portion 729 of the pumping chamber 725, thereby oxygenating the preservation solution. Because the oxygen will expand to fill the first portion 727 of the pumping chamber 725, substantially all of an upper surface 741 of the membrane 740 which faces the first portion of the pumping chamber can be used to diffuse the oxygen from the first portion into the second portion 729 of the pumping chamber.

As the tissue consumes oxygen, the tissue will release carbon dioxide into the preservation fluid. Such carbon dioxide can be diffused from the second portion 729 of the pumping chamber 725 into the first portion 727 of the pumping chamber 725. Carbon dioxide within the first portion 727 of the pumping chamber is vented via a control line (not shown) to a valve (not shown), and from the valve through a vent line (not shown) to the atmosphere external to the first transport container. The positive pressure also causes the membrane 740 to flex, which transfers the positive pressure in the form of a pulse wave into the oxygenated preservation fluid. The pulse wave generated by the pumping chamber is configured to facilitate circulation of the oxygenated preservation fluid from the second portion 729 of the pumping chamber 725 into storage chamber 792 thereby contacting the tissue or being perfused through the tissue.

At least a portion of the preservation fluid contacting the tissue is received in the storage chamber 792. In some embodiments, the pulse wave is configured to flow through the preservation solution disposed in the storage chamber 792 towards the floor 793 of the canister 790. The floor 793 of the canister 790 is configured to flex when engaged by the pulse wave. The floor 793 of the canister 790 is configured to return the pulse wave through the preservation fluid towards the top of the storage chamber 792 as the floor 793 of the canister 790 is returned towards its original non-flexed position. In some embodiments, the returned pulse wave is configured to generate a sufficient pressure to open the valves 738A, 738B disposed at the highest positions in the storage chamber 792. In this manner, the returned pulse wave helps to move the valves 738A, 738B to their respective open configurations such that excess fluid (e.g., carbon dioxide released from the body fluid and/or the preservation fluid) can move through the valves from the storage chamber 792 to the pumping chamber 725. The foregoing cycle can be repeated as desired, including in any manner described above with respect to other apparatus described herein.

The lid assembly 710 is configured to be coupled to the canister 790. The lid assembly 710 includes handles 712, 713. The handles 712, 713 are each configured to facilitate coupling the lid assembly 710 to the canister 790, as described in more detail herein. Said another way, the handles 712, 713 are configured to move between a closed configuration in which the handles prevent the lid assembly 710 being uncoupled or otherwise removed from the canister 790, and an open configuration in which the handles do not prevent the lid assembly 710 from being uncoupled or otherwise removed from the canister. The handles 712, 713 are moveably coupled to the lid 720. Each handle 712, 713 can be pivotally coupled to opposing sides of a coupling mechanism disposed about the lid 720. For example, each handle 712, 713 can be coupled to the coupling mechanism via an axle (not shown). Each handle includes a series of gear teeth (not shown) configured to engage a series of gear teeth disposed on opposing sides of the lid 720 as the handles 712, 713 each pivot with respect to the coupling mechanism, thus causing rotation of the coupling mechanism. In some embodiments, the handles 712, 713 include webbing between each tooth of the series of gear teeth, which is configured to provide additional strength to the respective handle. In their closed configuration, the handles 712, 713 are substantially flush to the coupling mechanism. In some embodiment, at least one handle 712 or 713 includes an indicia indicative of proper usage or movement of the handle. For example, the handle 713 includes indicia (i.e., an arrow) indicative of a direction in which the handle portion can be moved. In some embodiments, the handles 712, 713 include a ribbed portion configured to facilitate a grip by a hand of an operator of the apparatus 700.

The canister 790 can be similar in many respects to a canister described herein. For example, the canister 790 may include includes a wall 791, a floor (also referred to herein as “bottom”) 793, and a compartment 794 defined on its sides by the wall and on its bottom by the floor. The compartment 794 can form a substantial portion of the organ chamber 792.

At least a portion of the canister 790 is configured to be received in the lid assembly 710 (e.g., the base 732). The canister 790 includes one or more protruding segments 797 disposed adjacent, or at least proximate, to an upper rim 795 of the canister. Each segment 797 is configured to protrude from an outer surface of the canister 790 wall 791. The segments 797 are configured to help properly align the canister 790 with the lid assembly 710, and to help couple the canister 790 to the lid assembly 710. Each segment 797 is configured to be received between a pair of corresponding segments 721 of the lid 720. A length of the segment 797 of the canister 790 is substantially equivalent to a length of an opening 860 between the corresponding segments 721 of the lid 720. In this manner, when the segment 797 of the canister 790 is received in the corresponding opening of the lid 720, relative rotation of the canister 790 and lid 720 with respect to each other is prevented. The canister 790 can include any suitable number of segments 797 configured to correspond to openings between protruding segments 721 of the lid 720. For example, in one embodiment, the canister 790 includes ten segments 797, each of which is substantially identical in form and function, spaced apart about the outer perimeter of the canister 790 adjacent the upper rim 795. In other embodiments, however, a canister can include less than or more than ten segments.

A gasket 752 is disposed between the base 732 and the upper rim 795 of the wall 791 of the canister 790. The gasket 752 is configured to seal the opening between the base 732 and the wall 791 of the canister 790 to substantially prevent flow of fluid (e.g., the perfusate) therethrough. The segments 797 of the canister 790 are configured to engage and compress the gasket 752 when the canister 790 is coupled to the lid 720. The gasket 752 can be any suitable gasket, including, for example, an O-ring.

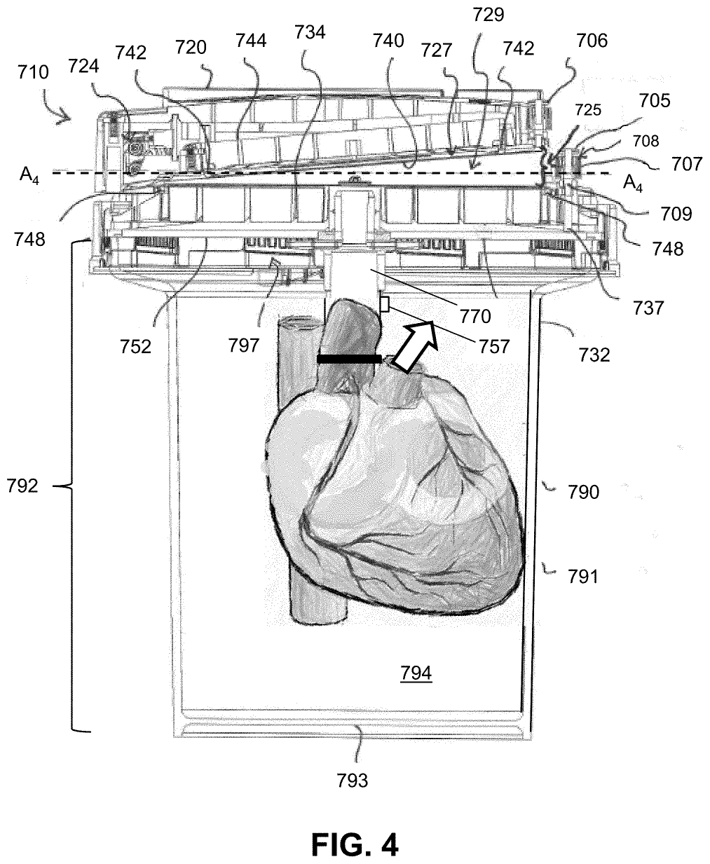

In some versions of the invention, the preservation solution is circulated through the tissue using the tissue's cardiovascular system. For example, as shown in FIG. 4, the tissue may be an organ, e.g., a heart. The tissue can be coupled to the pumping chamber via an adapter, which is shown in FIG. 4 as lumen 770. Lumen 770 may be directly attached to the organ, e.g., via the vena cava, allowing oxygenated preservation solution to be perfused through the organ. A temperature sensor 757 may also be affixed to lumen 770 and be used to monitor the temperature of the preservation fluid in close proximity to the tissue. As shown by the arrow in FIG. 4, the perfused preservation fluid will exit the organ, e.g., via a pulmonary artery, and return to the storage chamber 792. The circulation of the preservation fluid, described above, will allow the preservation solution to be re-oxygenated prior to being re-perfused into the tissue. Additionally, using a first transport container such as shown in FIG. 4, perfusion pressure can also be varied, e.g., once per second, between a low and a high pressure, thereby simulating the natural pulsatile flow of blood through the vasculature of the tissues. This method of perfusion provides a more “natural” environment for absorption of oxygen and nutrients from the preservation solution, increases the amount of time that the organ can be transported, and improves the overall quality of the tissue upon arrival. Furthermore, because compressed oxygen is used to propel the pulsed circulation, the preservation fluid is reoxygenated throughout transport, replacing the oxygen that has been consumed by the tissue and displacing waste gases (i.e., CO₂). In some versions, a suite of sensors measures temperature, oxygen content, and pressure of the circulating fluids to assure that the tissue experiences a favorable environment during the entire transport.

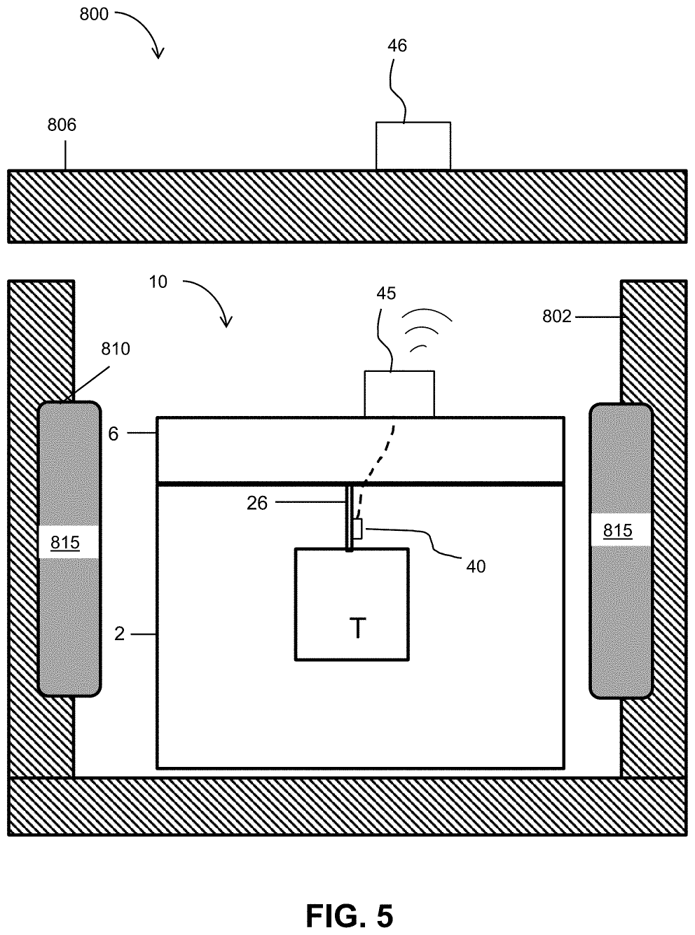

A complete system for hypothermic transport of tissues, comprising a static first transport container 1 and a second transport container 800 is shown in FIG. 5. The first static transport container comprises a storage vessel 2 and a lid without a pumping chamber 6, as described above. The second transport container 800 comprises an insulated vessel 802 and an insulated lid 806. The insulated vessel has at least one recess 810 configured to hold a cooling medium 815. As shown in FIG. 5, a sealed static first transport container 1 is placed in insulated vessel 802 along with cooling media 815, and the insulated lid is placed on insulated vessel 802 forming a temperature-regulated environment for transport of tissue.

The insulated vessel 802 and the insulated lid 806 will both comprise an insulating material that is effective in maintaining the temperature inside the second transport container 800. A suitable insulating material may be any of a number of rigid polymer foams with high R values, such as polystyrene foams (e.g. STYROFOAM™), polyurethane foams, polyvinyl chloride foams, poly(acrylonitrile)(butadiene)(styrene) foams, or polyisocyanurate foams. Other materials, such as spun fiberglass, cellulose, or vermiculite could also be used. Typically, the insulating vessel 802 will be constructed to provide a close fit for the first transport container, thereby affording additional mechanical protection to the first transport container and the tissues contained therein. In some embodiments, the insulated vessel 802 and the insulated lid 806 will be constructed of a closed-cell foam that will prevent absorption of liquids, for example water, body fluids, preservation fluid, saline, etc. While not shown in FIG. 5, the insulated vessel 802 and the insulated lid 806 may have a hard shell on the exterior to protect the insulating material from damage or puncture. The hard shell may be formed of metal (e.g. aluminum or steel) or of a durable rigid plastic (e.g. PVC or ABS). The hard shell may have antibacterial properties through the use of antibacterial coatings or by incorporation of metal that have innate antibacterial properties (e.g. silver or copper).

While not shown in FIG. 5, the insulated vessel 802 and the insulated lid 806 may be connected with a hinge, hasp, clasp, or other suitable connector. The second transport container 800 may include an insulating seal to make to make an air- or water-tight coupling between the insulated vessel 802 and the insulated lid 806. However, the insulated lid 806 need not be sealed to the insulated vessel 802 for the second transport container 800 to maintain a suitable temperature during transport. In some embodiments, the insulated vessel 802 and the insulated lid 806 will be coupled with a combination lock or a tamper-evident device. The insulated vessel 802 and/or the insulated lid 806 may additionally comprise a handle or a hand-hold or facilitate moving the second transport container 800 when loaded with a first transport container (static 1 or pulsatile 10). While not shown in FIG. 5, in some embodiments, insulated vessel 802 will additionally have external wheels (e.g. castor wheels or in-line skate type wheels). The insulated vessel 802 may also have a rollaboard-type retractable handle to facilitate moving the system between modes of transport or around a hospital or other medical facility.

In some embodiments, such as shown in FIG. 5, the second transport container 800 will comprise a second temperature display 46 which can display a temperature measured by the temperature sensor 40 to a user. The second temperature display 46 may receive measurements of temperature within the static first transport container 1 via a wired or a wireless connection. In the embodiment shown in FIG. 5, an electronics package on the lid 6 is coupled to the temperature display 45 and comprises a wireless transmitter that communicates with a receiver coupled to the second temperature display 46. This configuration avoids a user having to make a connection between the temperature sensor 40 and the second temperature display 46 after the first static transport container 1 has been placed in the insulated vessel. The second insulated transport container 800 may additionally comprise displays for additional relevant information, such as time since harvest, pressure inside the first transport container (static 1 or pulsatile 10), partial pressure of oxygen, or oxygen consumption rate of the biological sample.

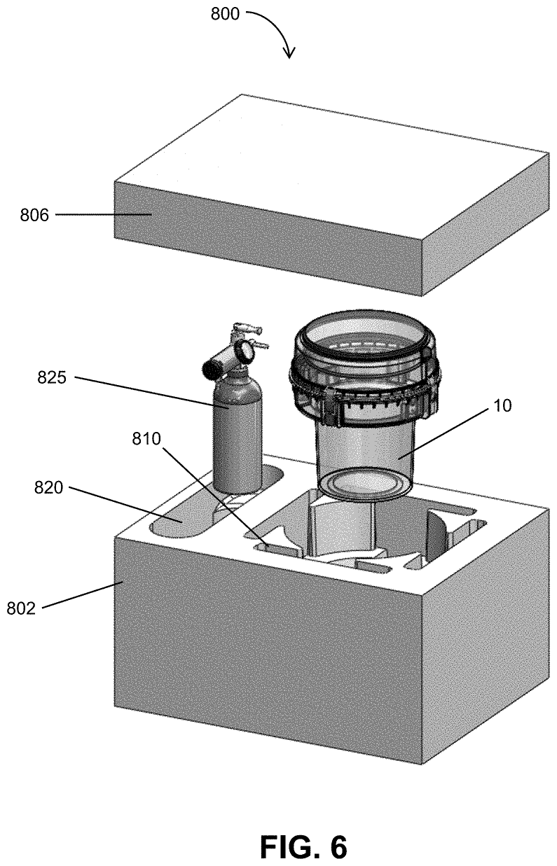

The system may use any of a number of cooling media 815 to maintain the temperature inside the second transport container 800 during transport. As shown in FIG. 5, the cooling media 815 may comprise eutectic cooling blocks, which have been engineered to have a stable temperature between 2-8° C., for example. The cooling media 815 will be arranged in recess 810 in the interior of the insulated vessel 802. The recess 810 may be a slot 825, such as shown in FIG. 6, or the recess may be a press-fit, or the cooling media 815 may be coupled to the walls of the insulated vessel 802 using a snap, screw, hook and loop, or another suitable connecter. Eutectic cooling media suitable for use with the invention is available from TCP Reliable Inc. Edison, N.J. 08837, as well as other suppliers. Other media, such as containerized water, containerized water-alcohol mixtures, or containerized water-glycol mixtures may also be used. The container need not be rigid, for example the cooling media may be contained in a bag which is placed in the recess 810. Using the cooling media 815, e.g. eutectic cooling blocks, the invention is capable of maintaining the temperature of the sample in the range of 2-8° C. for at least 60 minutes, e.g., for greater than 4 hours, for greater than 8 hours, for greater than 12 hours, or for greater than 16 hours.

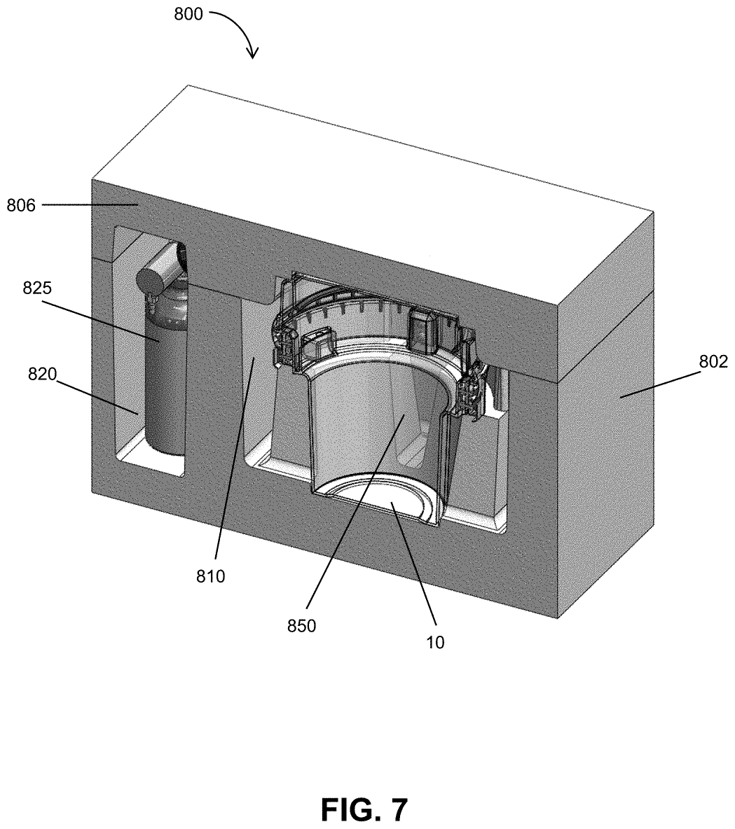

FIG. 6 shows another embodiment of a complete system for hypothermic transport of tissues, comprising a first transport container (1 or 10) and a second transport container 800. As in FIG. 5, the second transport container comprises an insulated vessel 802 and an insulated lid 806. The insulated vessel has recesses 810 for holding cooling media 815. As shown in greater detail in FIG. 7, the insulated vessel is formed to closely fit the first transport container (10) to provide mechanical protection to the container and to assure that the container remains upright during transport. The insulated vessel 802 and the insulated lid 806 have hard sides for durability, and may have wheels (not shown) for ease of transport. As shown in FIG. 6, the insulated vessel 802 additionally comprises an oxygenate recess 820 for holding a compressed oxygenate 825, for example a cylinder of compressed oxygen. As discussed in greater detail above, the compressed oxygenate can serve a dual purpose of oxygenating the preservation solution and also providing pressure to circulate the preservation solution around or through the tissue. While not shown in FIG. 6, second transport container 800 may additionally comprise a regulator and tubing to connect the compressed oxygenate to the first transport container (10).

As shown in the cut-away view of the second transport container 800 in FIG. 7, both the insulated vessel 802 and the insulated lid 806 are designed to snugly fit the first transport container (1 or 10) to provide additional mechanical stability. While not visible in FIG. 7, the oxygenate recess 820 also provides a snug fit for the compressed oxygenate, which may be, for example, a size 4 cylinder of compressed gas. Also, as shown in FIG. 7, a thermal communication passage 850 may be provided (behind wall of first transport container) to allow better thermal flow between the cooling media 815 and the first transport container (10). In some instances, the interstitial space between the cooling media 815 and the first transport container 10 will be filled with a thermal transport fluid, such as water or an aqueous solution. In other instances, the interstitial space will be filled with air or another gas (e.g. dry nitrogen).

The disclosed systems provide a better option for transporting biological samples than the “picnic cooler” method. In one embodiment a medical professional will provide a hypothermic transport system of the invention, for example as shown in FIGS. 5-7, suspend a biological sample in preservation fluid within a first transport container, for example as shown in FIG. 1, and maintain the temperature of the preservation fluid between 2 and 8° C. for at least 60 minutes. Because the first transport container has a temperature sensor and a temperature display, it will be possible for the medical professional to monitor the temperature of the sample after it has been sealed inside the first transport container. Such temperature information will be critical in evaluating the status of the sample during transport and for identifying failures during transport. In embodiments having a second display on the second transport container, it will be possible to monitor the temperature of the sample without opening the second transport container, thereby maintaining the hypothermic environment within.

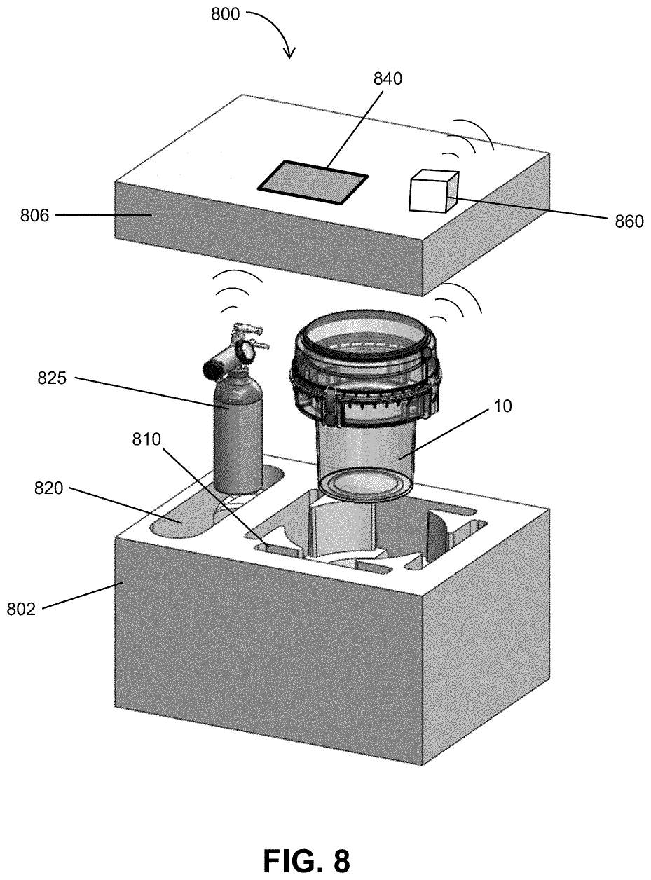

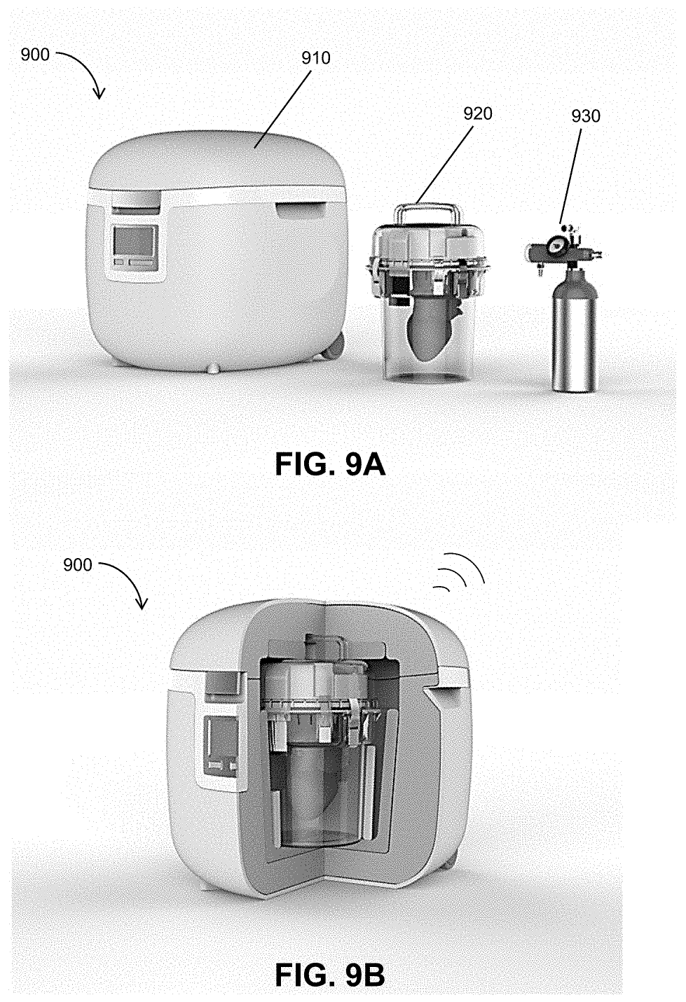

Using the systems of the invention, the preservation fluid may be maintained at a pressure greater than atmospheric pressure, and may be oxygenated, for example by an accompanying cylinder of compressed oxygen, i.e., as shown in FIG. 6. The cylinder of compressed oxygen may additionally include a sensor configured to measure the pressure of the oxygen within the cylinder and to transmit the pressure to a receiver 860, as shown in FIG. 8. In some embodiments the pressure readings will be displayed on display 840 on the second transport container. In some embodiments, the pressure data will be transmitted wirelessly to a network, so that the pressure data can be remotely monitored. An alternative embodiment of a hypothermic tissue transport system 900 of the invention is shown in FIGS. 9A and 9B, including the second transport container 910, the first transport container 920, and the source of compressed oxygen 930. As shown in FIG. 9B, all of the components can be assembled into a compact, and easily-transported package.

In some instances, the preservation fluid will be circulated around tissue suspended in the first transport container, or the preservation fluid may be perfused through an organ suspended in the first transport container. Preferably, an organ will be perfused with preservation solution by using oscillating pressures, thereby simulating the systolic and diastolic pressures experienced by circulatory system of the organ in the body. When body fluids are transported, the body fluids may be transported by suspending a third container (e.g., a blood bag) within the first transport container.

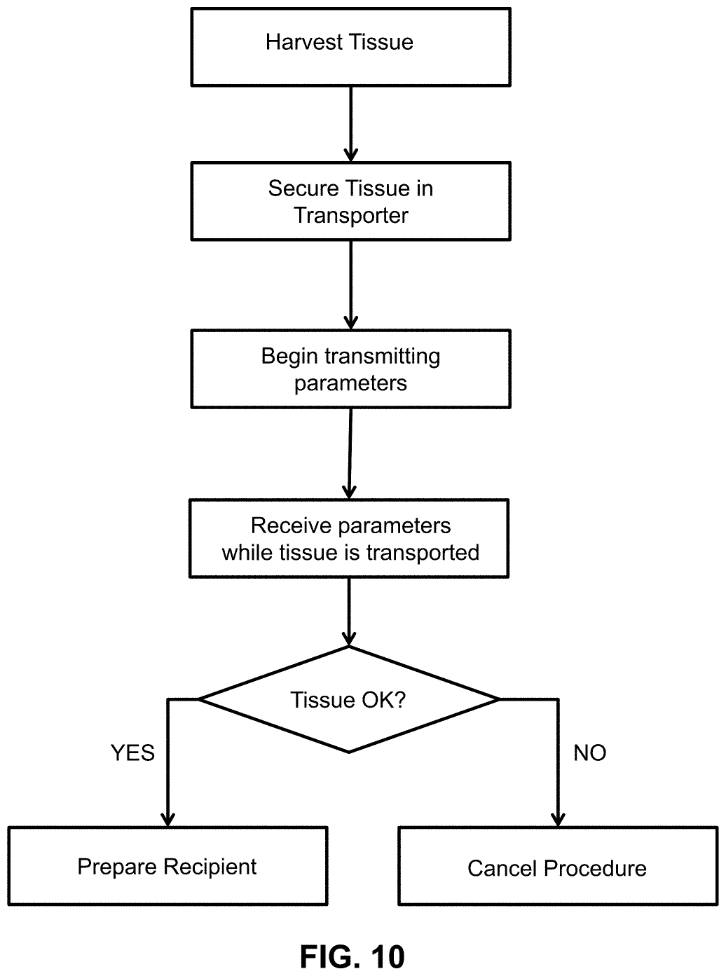

A flowchart illustrating the advantages of a system of the invention is shown in FIG. 10. Initially, the tissue is harvested. The tissue may be an organ or some other tissue such as skin tissue. Once the tissue has been secured in the transporter, the transporter will begin to transmit parameters, such as position, temperature, pressure, oxygen flow, and oxygen consumption. Throughout transport, this information can be received remotely by the transplant team, thereby allowing them to determine if the procedure should go forward. This feature is particularly important because tissue transport systems such as Sherpa™ (Paragonix Technologies, Braintree, Mass.), which incorporate active oxygen perfusion, can extend transport times up to 12 hours. Thus, it would be inappropriate to begin preparing a recipient at the time the organ is harvested.

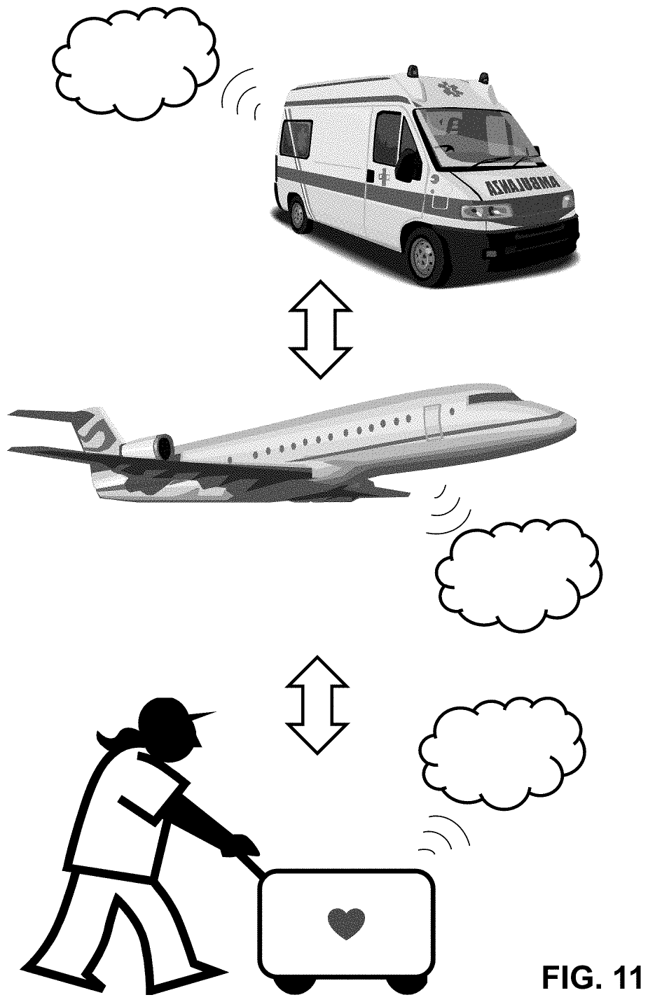

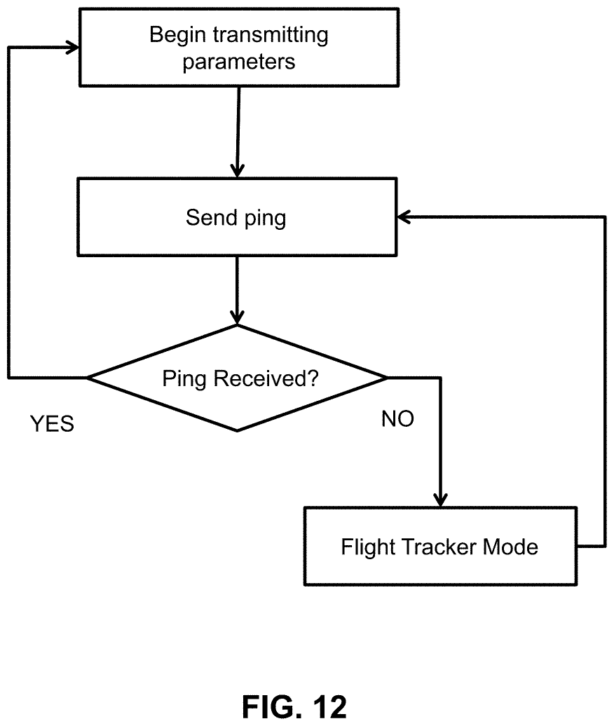

The active tracking features of the invention can be used to monitor the condition and position of a tissue regardless of the mode of transportation, as illustrated in FIG. 11. In some embodiments, the second transport container will be configured with multiple transmitters, allowing the signals to be handed off from mobile networks, i.e., 4G, to WiFi, to Bluetooth, depending upon the best available source of internet connectivity. In certain instances, wireless connectivity will be blocked because of safety concerns, such as during take-off and landing of an airplane. In an embodiment, the system is configured to sense when it has moved into a shielded environment, e.g., inside an aircraft or airport. As shown in FIG. 12, the system is configured to sense when it is no longer able to access the network, at which point the system will switch to flight-tracking to allow the receiving medical team to know the position of the system in real time. In other embodiments, the flight-tracking will be augmented with a continuous data stream of organ parameters, available via in flight WiFi.

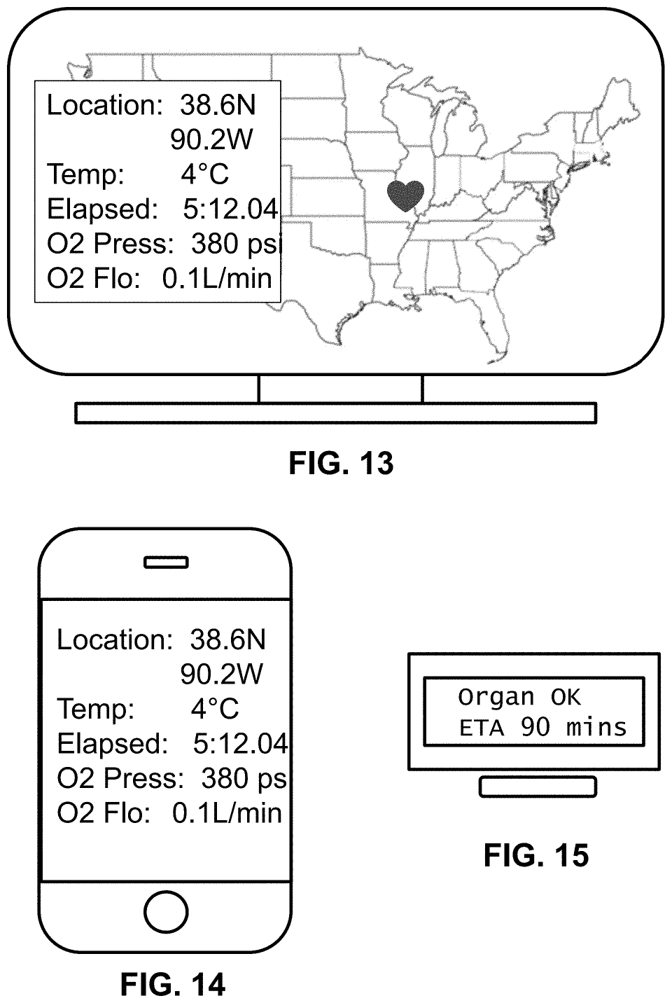

A number of options for receiving and displaying the information from the system are available, including direct networks, webpages, dummy terminals, mobile devices (smart phones), tablets, pagers, and smart watches, as shown in FIGS. 13-15. The data can be provided in a variety of ways, including text, maps, colors, and sounds.

Thus, using the system for hypothermic transport of tissues of the invention, it is possible to transport a biological sample (e.g. tissue, organs, or body fluids) over distances while maintaining a temperature of 2-8° C. Systems of the invention will enable medical professionals to keep tissues (e.g. organs) in a favorable hypothermic environment for extended periods of time, thereby allowing more time between harvest and transplant. As a result of the invention, a greater number of donor organs will be available thereby saving lives.

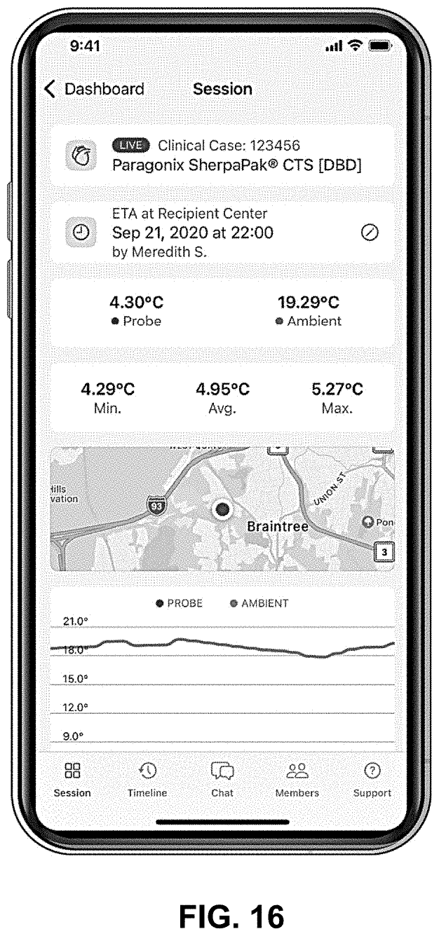

As shown in FIG. 16, a user interface on a computing device running a program as described herein may display organ status information. Exemplary data received from the transport team or connected transport device can include a case identifier, a real-time updated estimated time of arrival for the organ at the recipient location, and a current organ and external temperature (e.g. and provided via connected temperature probes inside and outside the organ container. The ETA may be set by a user via the user interface and the member who set the ETA can be identified on the session homepage. A status interface may also include minimum, maximum, and average temperatures recorded during the session (internal, external/ambient, or both) as well as current location via an identifier on a map. After appropriate time stamps are logged, an ischemic time can be displayed as well. Ischemic time may be calculated in a variety of manners depending on the organ being transported. For example, for a heart, it may be calculated as the time from the donor crossclamp timestamp to the recipient off claim timestamp. For a lung, ischemic time may be calculated as the time between donor crossclamp timestamp and a final (either left or right) anastomosis complete timestamp. For liver, the calculation may be from donor crossclamp timestamp to a final (either portal or arterial) perfusion complete timestamp. Graphical representations of temperature may be provided as well. Data can be updated in real time or in set intervals such as at least every 30 seconds or less, at least every minute or less, or at least every 5 minutes or less. The user interface may include tabs or icons for other features such as timeline, chat/communications, member identification, and user support.

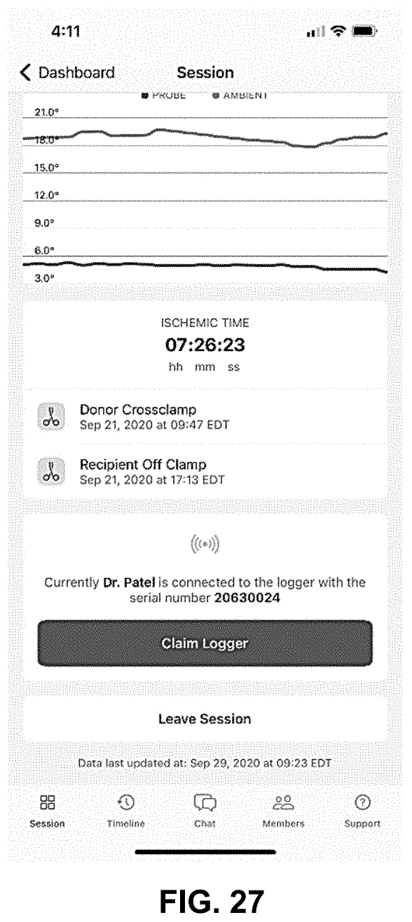

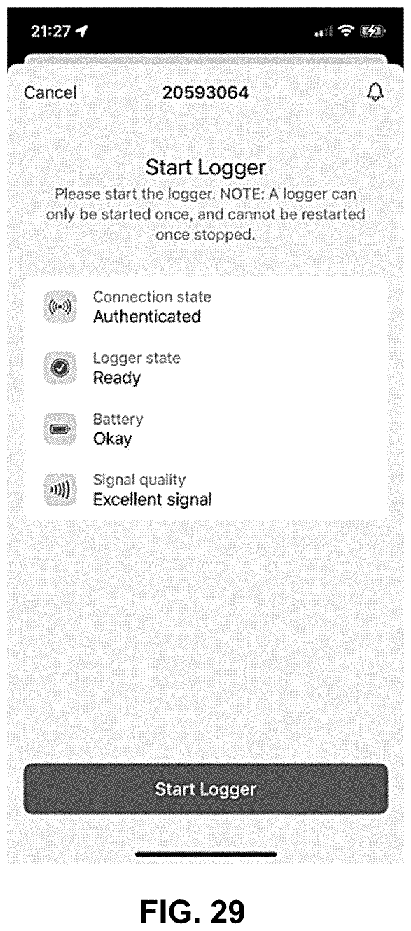

Additional information may include access to archived sessions as well as prompts to set session ETA, connect to a device logger (e.g., claim or disconnect to a device logger) and leave or stop a session as shown in FIG. 27. In various embodiments, only the session creator may have permission to end the session. In certain embodiments, only a single user can claim the logger at any point. Claiming the logger may include pairing to the transport device via Bluetooth or other connection protocol. The user may be automatically disconnected if they are out of range of the connection. An exemplary connection user interface is shown in FIG. 29. For examples, to connect to a transport device logger, a user can select “Connect Device” or a similar prompt or icon. The application can then scan for nearby transport devices. The user can then be prompted to confirm the correct logger utilizing the serial number for reference and/or be prompted to enter a passkey or code found on a label or otherwise affixed to the transport device. In certain embodiments, once entered the Passkey Code may be stored in the session in order for other session members to easily connect to the device. In various embodiments, device logger connections may be limited to one, two, three, five, ten, or less mobile devices at one time. The user can start the logger via a prompt in the user interface or may be started directly on the transport device through interaction with a keypad, touch screen, or other input device. For consistency, in certain embodiments, a logger may only be started once and cannot be restarted once stopped (e.g., cannot be paused).

In various embodiments, logger connection can be managed according to one or more of the following exemplary protocols. All session members may connect to the Logger, one at a time; Logger information and the current session Member connected to the Logger can be displayed on the Session Homepage; Members not connected to the Logger may see an option to Claim Logger on the Session Homepage; a Member currently connected to the Logger may see an option to Disconnect Logger on the Session Homepage; A Logger may only be claimed when no other Member is connected to the Logger or when the current Member connected to the Logger is out of Bluetooth connection range; If a connected Member is out of Bluetooth connection range and then returns to range, the connection may be automatically restored.

A session may be ended through the application user interface or directly via the transport device in various embodiments. Once the logger is stopped, all or some selected subset of the members may have access to download a session report containing the logged data.

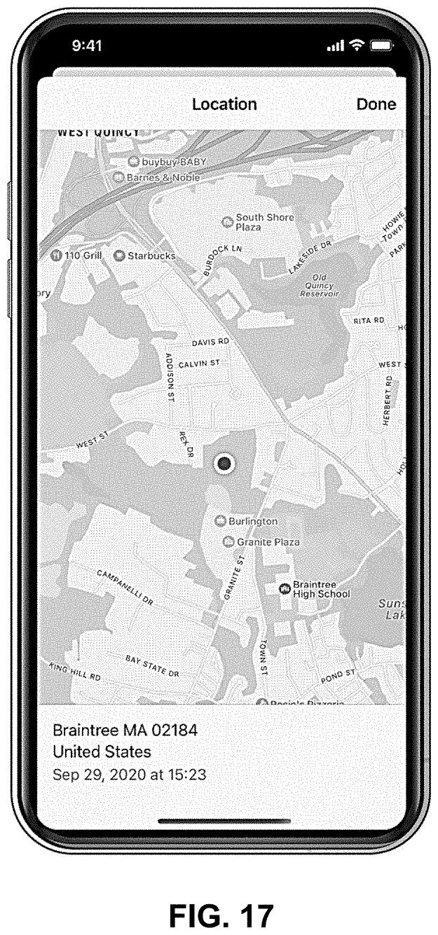

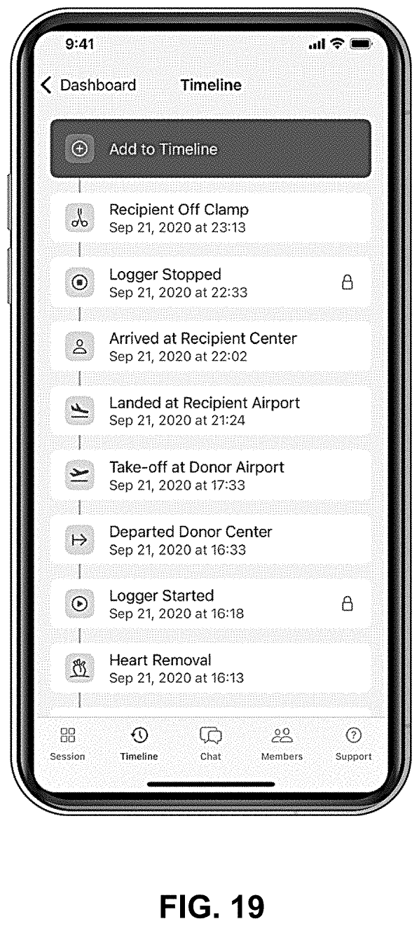

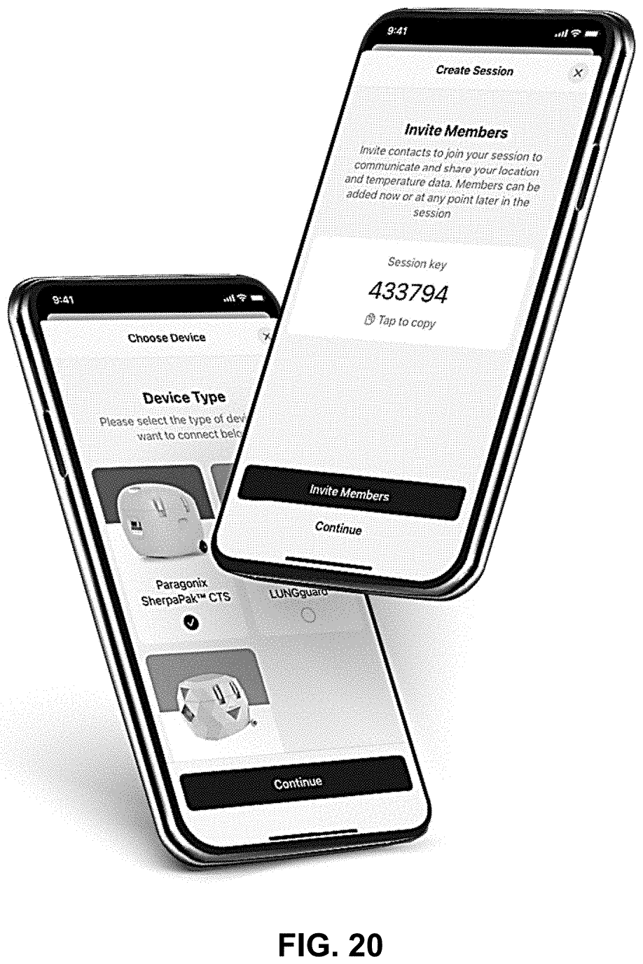

As shown in FIG. 17 a user interface can display organ location on a dedicated page which may include a graphical representation of the current location overlayed on a satellite or road map image. The location can be provided via GPS tracking of a sensor in or attached to the transport device or in a paired device such as a mobile phone possessed by a selected transport team member. FIG. 19 shows an exemplary user interface displaying case status that can include timestamps of critical logistical and clinical milestones. Those timestamps can be linked to pre-selected events and saved to provide a record of the organ procurement mission to aid in improving logistics, which could help clinical outcomes, by reducing total ischemic times. The timestamp log may be downloaded in certain embodiments, to create a permanent record for audits. In certain embodiments, case specific timestamps can be added by the user for important events by, for example, selecting an icon to add to timeline. In certain embodiments, a list of prepopulated time stamps may be provided for a user to add. In various embodiments, all session members or a selected subset of members may have permission to add timeline events. Information including the user that added the event may be recorded and shown with the timeline event. In certain embodiments, an Ischemic Time Calculation can be made based on recorded timeline events and reported in the session data.

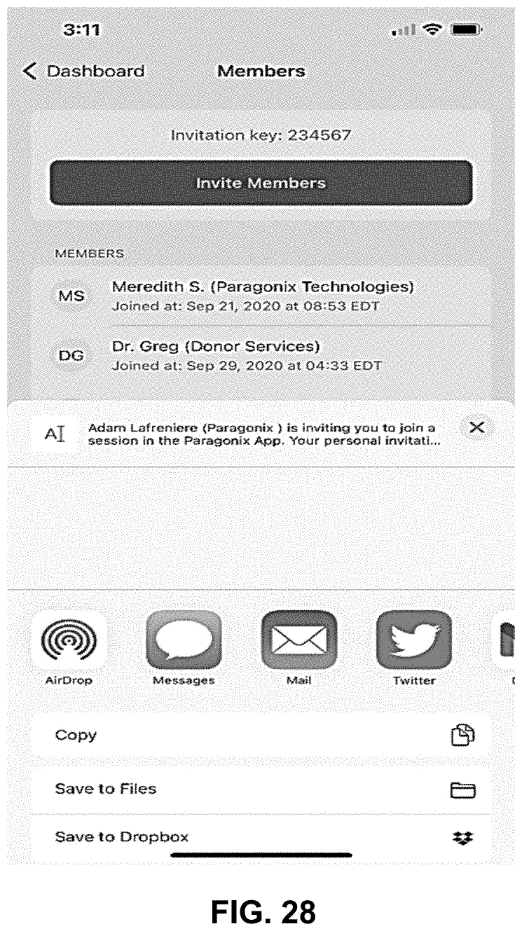

A member tab may be available to provide a user interface such as shown in FIG. 28 listing the members in the session, an option to invite members, the required invitation key to join the session and member information such as the date and time they joined the session.

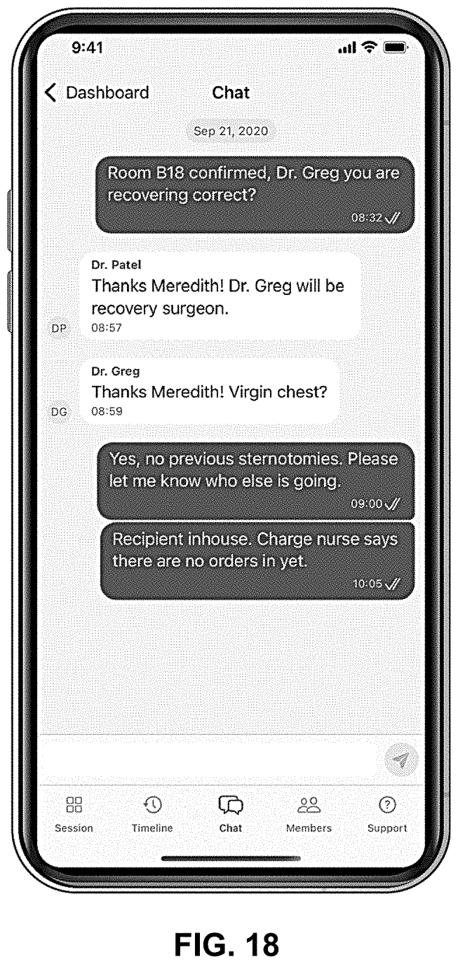

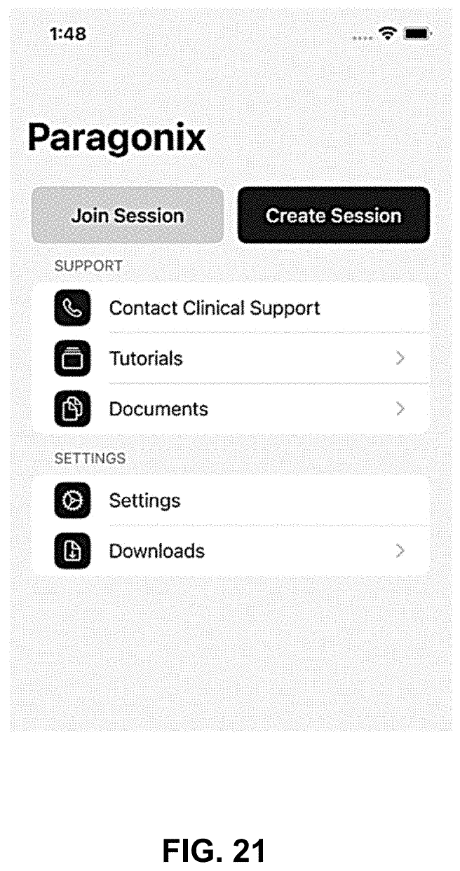

FIG. 18 shows an exemplary user interface displaying case team communications. Secure messaging systems can be provided for HIPAA-compliant chat for anyone that is invited to the session. Accordingly, such systems can eliminate the risk of non-compliance of CMS-mandated secure messaging or communication among transplant and OPO teams. The application can strip all patient related information (or restrict entry of personal information in the first place). Chat information can be manually or automatically purged/deleted upon session closure to ensure privacy. In certain embodiments, indications may be provided in the chat when a message has been successfully sent, received, and/or seen by all members of the session (e.g., one or two checkmarks of different colors). Upon interacting with the application (e.g., opening an application on a mobile device, running a program on a computer, or accessing a web portal) a user may be presented with a user interface such as shown in FIG. 21. The user may elect, for example, to join an existing session or create a new session. Access to support such as a clinical support contact (e.g., text or voice communication with a representative) can be provided along with access to tutorials, documents and settings. The session can be created preferably upon acceptance of a donor organ.

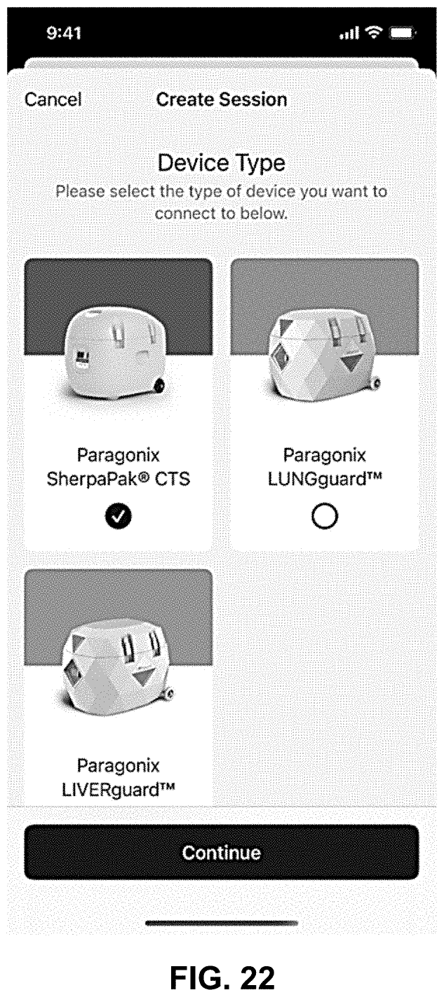

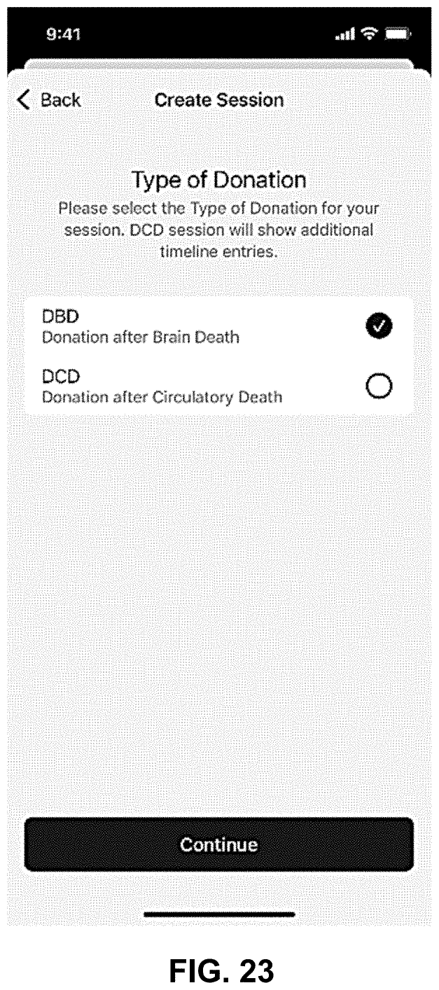

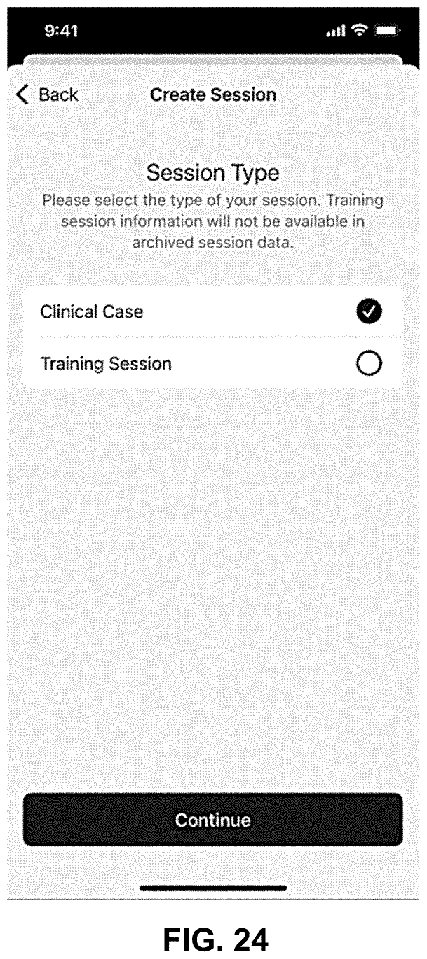

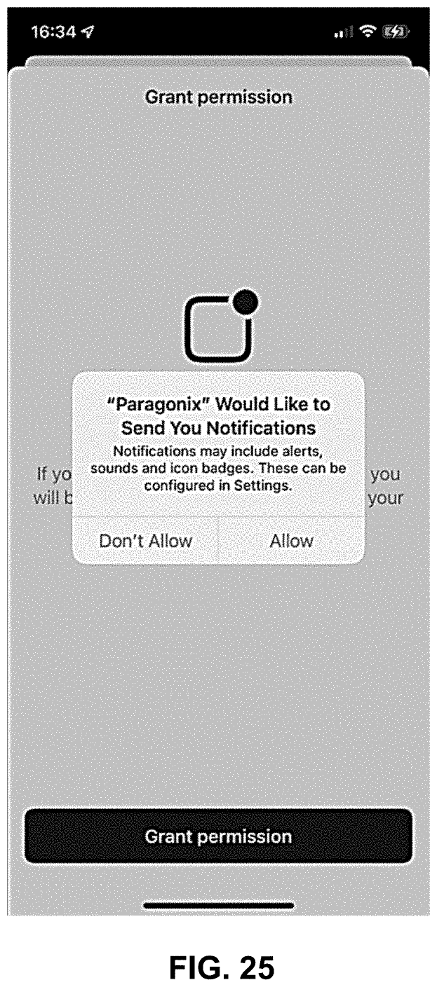

In creating a session, a user may be prompted to select a device type and may be provided, as illustrated in FIG. 22, with visual representations of various devices to aid in correct selection. A user can also be prompted for the type of donation (e.g., donation after brain death or donation after circulatory death) as shown in FIG. 23. Furthermore, a user may select a session type to allow for training sessions as shown in FIG. 24. Subsequent timeline events provided to users may be determined based on the type of device and/or donation selected. Data from training cases may be archived for reference in certain embodiments but segregated from clinical case data so as not to impact calculations of archive and web portal averages and not to impact research relying on transport data. Upon creating a session, a user can be prompted to allow notifications as shown in FIG. 25. The user can also be prompted to invite members which may include accessing the user's contacts.

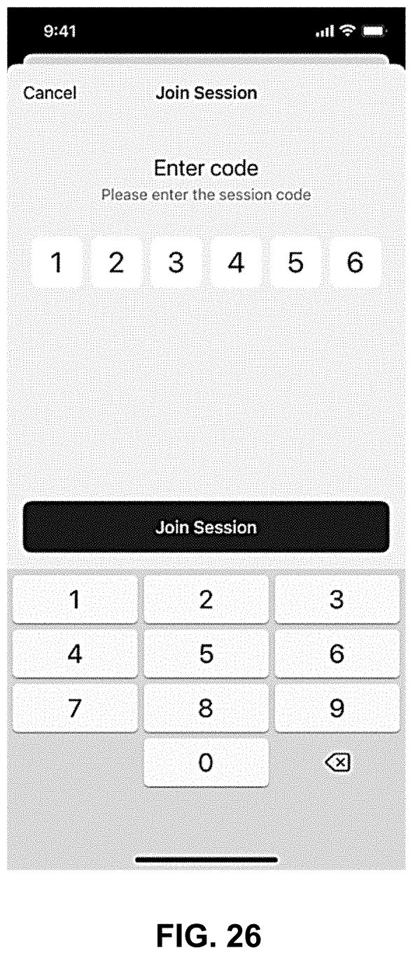

To join an existing session, for security, a user may be prompted to enter a session key as illustrated in FIG. 26. The session key may be shared by the session creator or may be provided on the device.

In various embodiments, data from current and/or archived sessions may be accessed and viewed via a web portal. Sessions may be accessed by providers (e.g., a donor or recipient hospital may have access to all current and archived sessions with which they were involved) for real time tracking or historical analysis. In certain embodiments, data can be sorted and/or filtered and may be anonymized and/or exported for research purposes and to comply with privacy requirements.

In certain embodiments, a dashboard user interface may allow a user to access critical case information; review latest sessions; sort by session key, date, device state, organ type, or avg. temp; track live sessions; or view historical session data in aggregate or by product including average temperature, average distance, and average ischemic time. Accessing an individual session can provide the user with all data from that session including timeline events, device information, and/or support and, in the case of live sessions, can provide information such as real-time temperature conditions, session eta, latest timeline event, GPS location, temperature vs time graph, and session and logger information.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A hypothermic tissue transport system comprising:
a tissue container;
a positioning transmitter;
at least one temperature sensor operable to provide temperature information for an interior cavity of the tissue container;
a computer device comprising a processor and a tangible, non-transitory memory, the computer device in wireless communication with the positioning transmitter and the at least one temperature sensor and operable to record temperature and position data therefrom; and
an output device in communication with the computer device and operable to display the recorded temperature and position data.

2. The hypothermic tissue transport system of claim 1, further comprising at least one of a pressure sensor, an oxygen sensor, an accelerometer, and a clock.

3. The hypothermic tissue transport system of claim 1, wherein the tissue is cardiac, epidermal, pulmonary, neurologic, nephrologic, or hepatic tissue.

4. The hypothermic tissue transport system of claim 1, wherein the positioning transmitter is a global positioning system (GPS) transmitter.

5. The hypothermic tissue transport system of claim 1, further comprising a wireless data transmitter.

6. The hypothermic tissue transport system of claim 5, wherein the wireless data transmitter uses a protocol selected from the group consisting of 3G, 4G, 4G LTE, 5G, WIFI, BlueTooth, WirelessHD, WiGig, Z-Wave, or Zigbee.

7. The hypothermic tissue transport system of claim 5, wherein the wireless data transmitter is a direct satellite data transmitter.

8. A system for monitoring the heath of a tissue during transport comprising:
a hypothermic tissue transport apparatus comprising a positioning receiver and a positioning transmitter;
a positioning network configured to receive a position of the hypothermic tissue transport apparatus;
a distributed network configured to transmit the position of the hypothermic tissue transport apparatus; and
an interface for displaying information about the position of the hypothermic tissue transport apparatus.

9. The system of claim 8, wherein the hypothermic tissue transport apparatus is configured to communicate with the distributed network wirelessly.

10. The system of claim 9, wherein the hypothermic tissue transport system is configured to measure pressure data, temperature data, acceleration, oxygen flow data, or oxygen consumption data and communicate said data to the distributed network.

11. The system of claim 10, wherein the interface is further configured to display information about pressure data, temperature data, acceleration, oxygen flow data, or oxygen consumption data.

12. The system of claim 11, wherein the system is configured to access flight data when the hypothermic tissue transport apparatus is being transported by an aircraft.
